(19) United States
(12) Patent Application Publication
(10) Pub. No.: US 2012/0178710 A1
(43) Pub. Date: Jul. 12, 2012

(54) SYNTHESIS OF CYCLIC DIGUANOSINE MONOPHOSPHATE AND THIOPHOSPHATE ANALOGS THEREOF

(75) Inventors: Roger A. Jones, New Brunswick, NJ (US); Barbara L. Gaffney, New Brunswick, NJ (US)

(73) Assignee: Rutgers, The State University of New Jersey, New Brunswick, NJ (US)

(21) Appl. No.: 13/381,622

(22) PCT Filed: Jul. 1, 2010
(86) PCT No.: PCT/US10/40825

§ 371 Date: Mar. 21, 2012

Related U.S. Application Data
(60) Provisional application No. 61/358,261, filed on Jun. 24, 2010.
(60) Provisional application No. 61/246,021, filed on Sep. 25, 2009.
(60) Provisional application No. 61/222,214, filed on Jul. 1, 2009.

(51) Int. Cl. A61K 31/708; C07H 1/00; C07H 19/20

(52) U.S. Cl. 514/48; 536/26.12; 536/25.33; 536/25.31

(57) ABSTRACT

The invention provides methods for the synthesis of cyclic dinucleotides and thiophosphate analogs thereof as well as a new family of analogs of cyclic diguanosine monophosphate that includes a series of seven phosphorothioate derivatives that includes diastereomers of mono-, di-, and trithiophosphates.

### CROSS-REFERENCE TO RELATED APPLICATIONS

This patent application claims the benefit of priority of U.S. application Ser. No. 61/222,214, filed Jul. 1, 2009, U.S. application Ser. No. 61/246,021, filed Sep. 25, 2009, and U.S. application Ser. No. 61/358,261, filed Jun. 24, 2010, which applications are herein incorporated by reference.

### STATEMENT OF GOVERNMENT SUPPORT

This invention was made with government support under Grant # GM079760 awarded by the National Institutes of Health. The government has certain rights in the invention.

### BACKGROUND

The bacterial signaling molecule cyclic diguanosine monophosphate (c-di-GMP), is increasingly recognized as having widespread consequences for human health through its multiple roles.

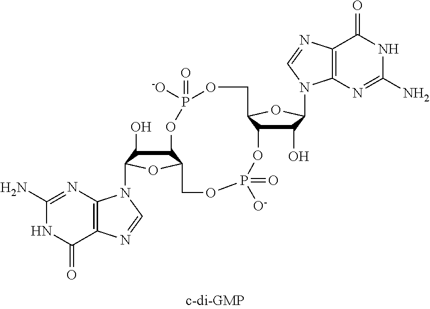

Not only is c-di-GMP a major factor in the activation of bacterial biofilm formation and repression of motility, it also helps to regulate virulence. Further, although c-di-GMP is not a signaling molecule in Eukarya, it has been shown to be an immunostimulatory agent that can trigger the innate immune response in mice. Yet the mechanisms for how c-di-GMP functions remain unclear. Accordingly, improved methods for producing c-di-GMP are needed as are analogs thereof.

### SUMMARY OF CERTAIN EMBODIMENTS OF THE INVENTION

Present herein is a new family of analogs of c-di-GMP: a series of seven phosphorothioate derivatives that includes diastereomers of mono-, di-, and trithiophosphates. The phosphorothioates described herein are expected to show significant activity and enhanced resistance to hydrolysis in a wide range of bacterial pathways that use c-di-GMP for signaling. Because such phosphorothioates are attractive candidates, synthetic methods for making them are needed, and an understanding of their behavior in solution is essential.

Seven phosphorothioate analogs of c-di-GMP (all diastereomers of mono-, di-, and trithiophosphates) were prepared to assess the impact of the thioate substitutions on c-di-GMP polymorphism using 1D ¹H and ³¹P NMR, along with 2D NOESY and DOSY, for both the Na⁺ and K⁺ salts. The K⁺ salts display more extensive higher order complex formation than the Na⁺ salts, resulting primarily in octamolecular complexes with K⁺, but tetramolecular complexes with Na⁺. Further, the presence of one or two [S_{P}] sulfurs specifically stabilizes anti complexes and/or destabilizes syn complexes, while the presence of two [S_{P}] sulfurs promotes extensive aggregation.

Also provided herein is an integrated set of reactions and conditions that allow a one-flask synthesis of the protected derivatives of c-di-GMP and the [Rₚ,Rₚ] and [Rₚ,Sₚ] thiophosphate analogs thereof.

### DETAILED DESCRIPTION

Accordingly, certain embodiments of the present invention provide a compound of formula 5a, 5b, 6a, 6b, 6c, 7a or 7b

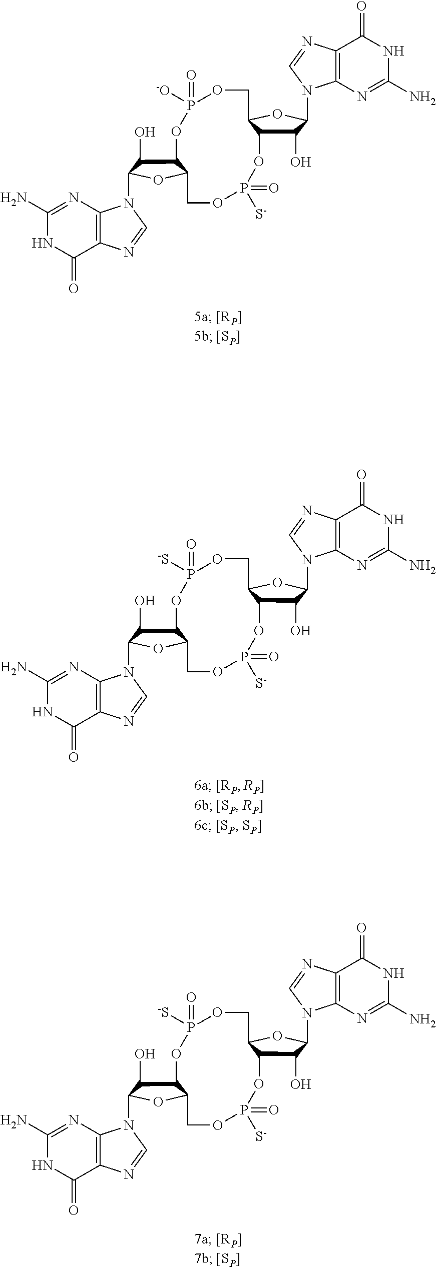

or a salt thereof.

In certain embodiments, the compound is compound 5a, or a salt thereof.

In certain embodiments, the compound is compound 5b, or a salt thereof.

In certain embodiments, the compound is compound 6a, or a salt thereof.

In certain embodiments, the compound is compound 6b, or a salt thereof.

In certain embodiments, the compound is compound 6c, or a salt thereof.

In certain embodiments, the compound is compound 7a, or a salt thereof.

In certain embodiments, the compound is compound 7b, or a salt thereof.

In certain embodiments, the salt is a potassium salt or a sodium salt.

In certain embodiments, the salt is a sodium salt.

In certain embodiments, the salt is a potassium salt.

Certain embodiments of the present invention provide a composition comprising a compound as described, or a salt thereof, and a carrier.

In certain embodiments, the carrier is a pharmaceutically acceptable carrier.

Certain embodiments of the present invention provide a method for preparing a protected derivative of a cyclic dinucleotide or a thiophosphate analog thereof, comprising converting a corresponding first and second phosphoramidite to the protected derivative of the cyclic dinucleotide or a thiophosphate analog thereof, which method comprises, in a single container:

hydrolyzing the first phosphoramidite;

coupling the second phosphoramidite to the hydrolyzed first phosphoramidite to provide a coupled compound;

oxidizing or sulfurizing the coupled compound to provide a linear dimer;

cyclizing the linear dimer;

isolating the protected derivatives;

optionally separating and purifying thiophosphate diastereomers; and

optionally deprotecting the cyclic dinucleotide or a thiophosphate analog thereof to provide the cyclic dinucleotide or a thiophosphate analog thereof.

In certain embodiments, the method comprises separating and purifying thiophosphate diastereomers.

In certain embodiments, the method comprises deprotecting the cyclic dinucleotide or a thiophosphate analog thereof to provide the cyclic dinucleotide or a thiophosphate analog thereof.

In certain embodiments, the coupled compound is oxidized to provide the linear dimer.

In certain embodiments, the coupled compound is sulfurized to provide the linear dimer.

In certain embodiments, each of the first and second phosphoramidites is individually selected from an adenosine, guanosine, cytidine, uridine, inosine, 5-methyl uridine, 5-bromouridine, 5-iodouridine, 6-thioguanosine, 2-aminopurine, 7-deazadenosine, zebularine, 8-aza-7-deazadenosine, pseudouridine, and 2,6-diaminopurine phosphoramidite, and a 2′-fluoro analog of an adenosine, guanosine, cytidine, or uridine phosphoramidite.

In certain embodiments, each of the first and second phosphoramidites are phosphoramidites of the same compound.

In certain embodiments, each of the first and second phosphoramidites are phosphoramidites of different compounds.

In certain embodiments, the first and second phosphoramidites are guanosine phosphoramidites.

In certain embodiments, the method provides a compound of formula 7, or a salt thereof, as a product

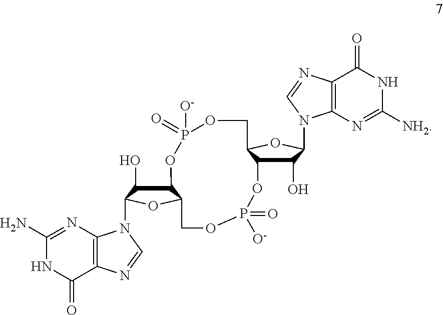

In certain embodiments, the method provides a compound of formula 8a or 8b, or a salt thereof, as a product

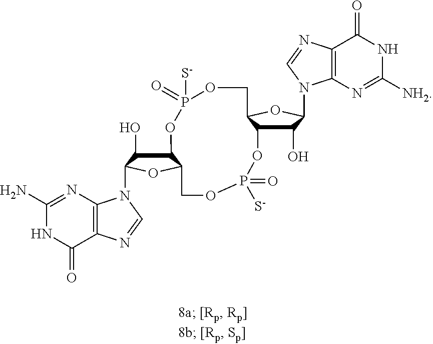

In certain embodiments, the container is a flask.

Certain embodiments of the present invention provide a compound prepared according to the method described herein.

Certain embodiments of the present invention provide compound as described herein for use in medical treatment or diagnosis.

Certain embodiments of the present invention provide a compound as described herein for use in therapy.

The approach to synthesize cyclic dimers described herein is not limited to a particular nucleoside or protecting group(s) (e.g., amino, phosphorous, or hydroxyl protecting groups). The most directly applicable monomers, specifically referred to in Example 2, are any of the commercially available phosphoramidites. There are a large number of available phosphoramidites with the same or similar protecting groups, and there are others where the 2′ protecting group differs. In addition to the standard nucleosides adenosine, guanosine, cytidine, and uridine, minor nucleosides such as, but not limited to, include inosine, 5-methyl uridine, 5-bromouridine, 5-iodouridine, 6-thioguanosine, 2-aminopurine are available, 7-deazadenosine, zebularine, 8-aza-7-deazadenosine, pseudouridine, 2,6-diaminopurine, as well as the 2′-fluoro analogs of A, G, C, and U (available from Glenn Research, as well as other vendors) with similar protecting groups as described herein. Accordingly, phosphoramidites of such nucleosides can be used in the methods described herein, e.g., in Example 2. In addition, the dimer does not have to contain the same two nucleosides, e.g., one could prepare cyclic AMP-GMP.

The invention will now be illustrated by the following non-limiting Examples. The numbering of compounds in each Example is specific to that Example (i.e., compound 1 in Example 1 is not necessarily the same compound as compound 1 in Example 2).

### Example 1

### Thiophosphate Analogs of c-di-GMP: Impact on Polymorphism

The bacterial signaling molecule, cyclic diguanosine monophosphate (c-di-GMP), is increasingly recognized as having widespread consequences for human health through its multiple roles. Not only is c-di-GMP a major factor in the activation of bacterial biofilm formation and repression of motility, it also helps to regulate virulence. Further, although c-di-GMP does not appear to be a signaling molecule in Eukarya, it has been shown to be an immunostimulatory agent that can trigger the innate immune response in mice. Yet the mechanisms for how c-di-GMP functions remain unclear. Studies using a series of analogs of c-di-GMP should facilitate elucidation of its modes of action, and would also allow evaluation of their potential use in the design of new therapeutic agents.

In addition to a self-intercalated bimolecular structure, c-di-GMP can adopt four different but related higher order guanine quartet structures, all in slow equilibrium. The analog in which one guanine residue is replaced with hypoxanthine (c-GMP-IMP) does not adopt these higher order complexes, presumably because the absence of even one guanine amino group precludes quartet formation.

A new family of analogs of c-di-GMP is described herein: a series of seven phosphorothioate derivatives that includes all diastereomers of mono-, di-, and trithiophosphates. All of the phosphorothioates described here are predicted to show significant activity and enhanced resistance to hydrolysis in a wide range of bacterial pathways that use c-di-GMP for signaling, with some diastereomers likely more effective than others. Because such phosphorothioates are attractive candidates, synthetic methods for making them are needed, and an understanding of their behavior in solution is essential.

The K⁺ salts of all seven thioates display more extensive higher order complex formation than do the Na⁺ salts. None-the-less, 1D ³¹P NMR always shows small amounts of the bimolecular structure in the K⁺ salts, even when the less sensitive 2D DOSY cannot detect it. Although K⁺ and Na⁺ ions are both known to stabilize guanine quartets in nucleic acids, in general the larger and more easily dehydrated K⁺ does so more effectively. K⁺ has frequently been found to be located between the planes of the quartets, whereas Na⁺ is often within the planes. It is perhaps for this reason that K⁺ can better stabilize the stacked quartet structures of c-di-GMP and its analogs, thereby promoting octamolecular complexes. Without this additional stabilization, tetramolecular complexes are dominant with Na⁺.

The presence of sulfur in the [S_{P}] configuration (5b, 6b, 6c, 7a, and 7b) specifically stabilizes anti complexes and/or destabilizes syn complexes in the Na⁺ salts, to the extent that no syn complexes are observed. However, for the K⁺ salts, both syn and anti complexes are present. Examination of the x-ray structures of the bimolecular form shows that the [S_{P}] sulfur is directed more toward the interior of the molecule, where the guanine rings are stacked, than is the [R_{P}]. The [S_{P}] sulfur may therefore be better positioned to create stabilizing interactions with key parts of the anti complexes and/or destabilizing clashes with parts of the syn complexes for the Na⁺ salts.

In addition, for both the K⁺ and Na⁺ salts, the presence of two [S_{P}] sulfurs (6c and 7b) gives poor spectra, perhaps as the result of extensive aggregation. This aggregation may take the form of end-to-end stacking of the tetra- or octamolecular complexes, forming extended guanine quartet structures, although other forms of aggregation cannot be excluded. Some of the residual resonances in the K⁺ salts of 6c and 7b, which must reflect very low concentrations, given the poor signal to noise ratio, show diffusion coefficients that are unusually small (1.1-1.4), and may represent aggregates just small enough to be detected. It is noted that the preliminary NMR used thioate samples with 0.1 M salts. However, the aggregation was so extensive that just the stoichiometric amount was used, with no excess salt, for the work presented here.

Thus, seven thiophosphate analogs of c-di-GMP were synthesized by new routes, and 1D ¹H and ³¹P NMR, along with 2D NOESY and DOSY, were used to assess the impact of the thioate substitution(s) on the polymorphism of both the Na⁺ and K⁺ salts. The results demonstrate the dramatic effects of [S_{P}] thiophosphates, as well as major differences between the Na⁺ and K⁺ salts. In all cases, the K⁺ salts display more extensive higher order complex formation than the Na⁺ salts, resulting primarily in octamolecular complexes for the K⁺ salts, but tetramolecular complexes for the Na⁺ salts. Further, the presence of just one [S_{P}] sulfur is sufficient to shift the population of the complexes entirely to the anti form, with no detectable syn complexes. The presence of two [S_{P}] sulfurs promotes extensive aggregation. Phosphorothioate derivatives of c-di-GMP are likely to prove useful for further exploration of the mechanisms of c-di-GMP function, where an understanding of the very different behavior of these diastereomers may be valuable.

### Synthesis

The syntheses of diastereomers of the mono-, di-, and trithiophosphate analogs of c-di-GMP, 5-7, were carried out as shown in Scheme 4. The first step is coupling of the standard commercially available guanosine phosphoramidite, 1, with the guanosine H-phosphonate monoester, 2a, or the H-thiophosphonate monoester, 2b. After coupling, the new phosphite triester is selectively oxidized using tert-butyl hydroperoxide, or sulfurized using elemental sulfur, in either case without affecting the H-phosphonate or H-thiophosphonate monoester. Detritylation is carried out using sodium bisulfate adsorbed to silica gel. Approximately equal amounts of the diastereomers of 3b or 3c are produced by the sulfurization. The linear dimers 3a-c are then ready for cyclization by treatment with pivaloyl chloride or diphenylchlorophosphate. Oxidation using iodine and water or sulfurization using elemental sulfur gives the cyclic derivatives 4a-e. Sulfurization of the H-phosphonate diester produced by cyclization gives only the [R_{P}] diastereomer. Purification of the linear and cyclic dimers 3a-c and 4a-e was carried out on silica gel, normally without attempting to separate the diastereomers at this stage. The polar groups present, along with the mixtures of diastereomers, hampered the silica purification and lowered the yields. Deprotection of 4a-e was carried out by treatment with methylamine in H₂O to remove the isobutyryl and cyanoethyl protecting groups, followed by desilylation using triethylamine.trihydrogen fluoride. Purification of 5-7 was carried out by RP HPLC to isolate each of the diastereomers 5a,b, 6a,b,c, and 7a,b. The separations were difficult, requiring repeated chromatography to get pure diastereomers, with concomitant loss of material that in some cases was significant, such that the overall yields from 2 to 5-7 did not exceed 10%.

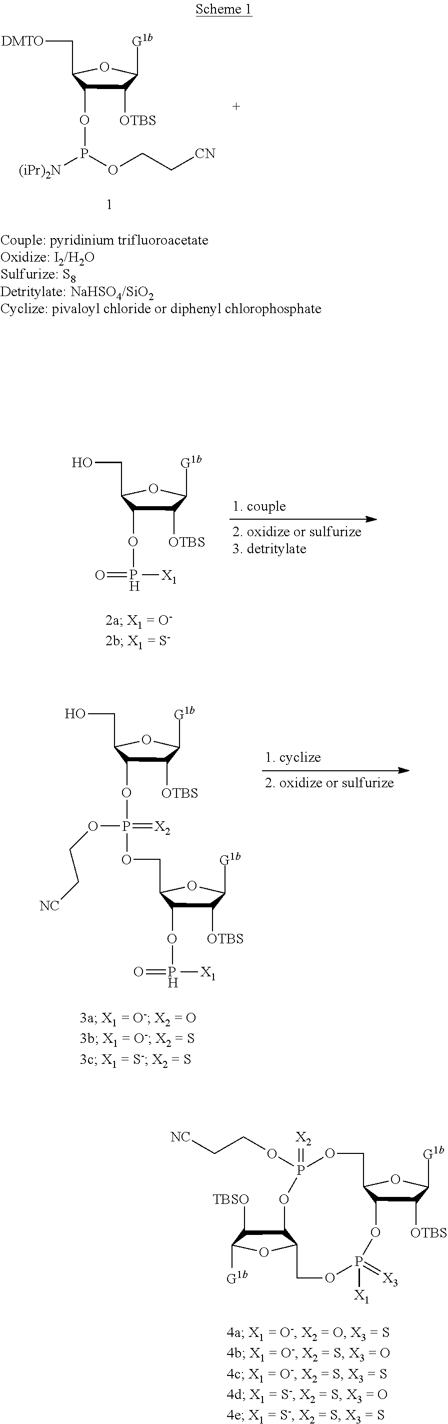

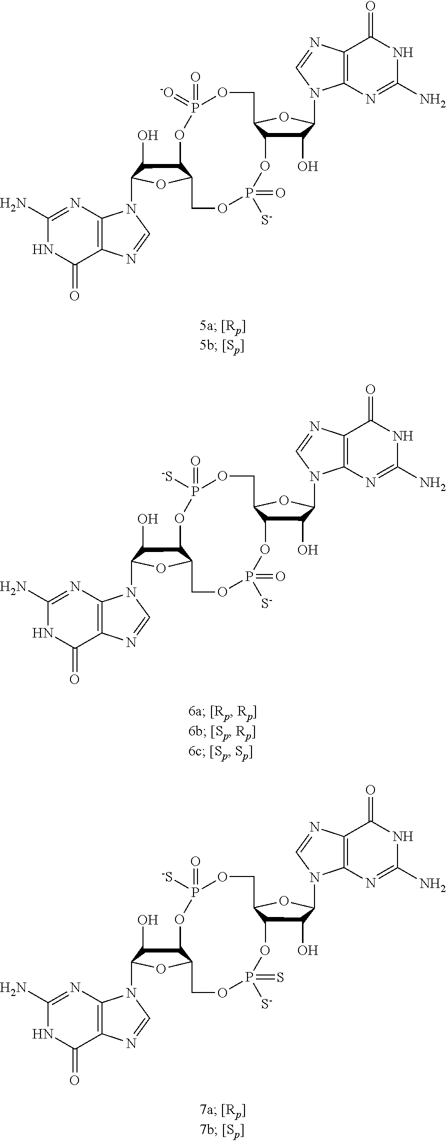

### Configuration Assignments

The only other work describing synthesis and characterization of stereochemistry of c-di-GMP monothiophosphates made assignments based on a comparison of the ³¹P NMR chemical shifts to those of the c-AMP phosphorothioates (c-AMPS), whose configuration had been established. The ³¹P NMR chemical shifts found are nearly identical to the chemical shifts reported for their c-di-GMP monothiophosphates, with about a 1.5 ppm difference between the two diastereomers. It was also found that the diastereomer with the larger ³¹P NMR chemical shift has the shorter retention time on RP HPLC. X-ray structures of c-di-GMP have shown the 2′-exo/3′-endo conformation typically found for RNA, and also found in c-AMP. The singlets in the ¹H NMR for the H1′ in c-di-GMP, and for all of the thioates, confirm that this is also the sugar conformation in solution. The 1.5 ppm difference in ³¹P NMR chemical shifts for the diastereomers is, however, well within the envelope of chemical shifts observed for the various complexes of c-di-GMP, and the thioate derivatives reported here. Further, the relationship between phosphate and nucleoside in the twelve-member ring of c-di-GMP is strikingly different from that in the six-member ring of c-AMP, as the x-ray structures demonstrate. In c-AMP, the phosphate is in a rigid chair conformation directly opposite C4′, while in the larger twelve-member ring of c-di-GMP the phosphates are moved well away from C4′. Given the small chemical shift differences between the diastereomers relative to the larger chemical shift changes for different c-di-GMP complexes, and the lack of similarity of the phosphate environment between c-di-GMP and c-AMP, the configuration of the thiophosphate diastereomers was examined using the now well established specificities of venom phosphodiesterase and nuclease P1. These enzymatic studies gave assignments of c-di-GMP thiophosphate configuration that are opposite to assignments based on c-AMPS. These enzymatic assignments are used for all of the compounds reported below.

The specificity of a variety of enzymes for differential cleavage of the diastereomers of linear 3′,5′-dinucleoside thiophosphates has been well known at least since Eckstein showed, by x-ray crystallography, that ribonuclease A gave exclusively the R_{P} diastereomer of uridine-3′-O-methyl ester from endo-uridine 2′,3′-cyclothiophosphate. The present approach to the configuration assignment of the c-di-GMP monothiophosphate diastereomers was to relate their configurations to those of the corresponding linear 3′,5′-diguanosine thiophosphates, where the configuration could be established by enzymatic cleavage by venom phosphodiesterase and by nuclease P1. Venom phosphodiesterase preferentially cleaves the R_{P} diastereomer, while P1 preferentially cleaves the S_{P} diastereomer. The results for the linear dimers were related to the cyclic dimers in two ways. First, as shown in Scheme 2, the linear dimers of 3b, before detritylation, (i) were separated by silica chromatography. A portion of each diastereomer (i) was dephosphitylated²⁶ and deprotected to give ii, which was characterized by cleavage with venom phosphodiesterase. The major portion of each separated diastereomer (i) was used for cyclization, followed by deprotection to give iii, which was characterized by ³¹P NMR and HPLC retention time. Thus a single linear dimer diastereomer i gave a single deprotected linear dimer ii and a single c-di-GMP monothiophosphate iii. The second approach was to work back from the mixture of the diastereomers of iii (prepared without prior separation of the linear diastereomers 3b) that were then separated by RP HPLC and characterized by ³¹P NMR and HPLC retention time. Because nuclease P1 cleaves phosphate diesters much faster than either of the thiophosphate diastereomers, brief treatment of iii with P1 was used to generate the corresponding linear dimer iv. As shown in Scheme 2, the linear dimers iv obtained from opening of iii have a 5′ phosphate. The presence of this phosphate should have no effect on the enzymatic cleavage of the thiophosphate. Nevertheless, a portion of iv was converted to ii by treatment of iv with bacterial alkaline phosphatase (BAP). The configuration of the linear dimers, ii and iv, obtained from iii, then were determined by treatment with venom phosphodiesterase as well as by treatment with nuclease P1. The results of the enzymatic cleavage experiments were identical for both pathways, regardless of the source of the dimer: the c-di-GMP monothiophosphate diastereomer having the longer HPLC retention time and the smaller thiophosphate ³¹P NMR chemical shift corresponded to the linear dimer that proved the better substrate for venom phosphodiesterase and the poorer substrate for nuclease P1, which must be the R_{P} diastereomer; conversely, the c-di-GMP monothiophosphate diastereomer having the shorter HPLC retention time and the larger thiophosphate ³¹P NMR chemical shift corresponded to the linear dimer that proved the poorer substrate for venom phosphodiesterase and the better substrate for nuclease P1, which must be the S_{P} diastereomer. The assignments of the di- and trithioate diastereomers were then made based on their retention times and chemical shifts, as shown in Table 1, with the smaller chemical shift/longer retention time assigned the R_{P} configuration.

TABLE 1
Retention Times on RP HPLC (min) and ³¹P NMR Chemical
Shifts (ppm) at 55° C. for Dilute (0.5 mM) Samples of the
Na⁺ Salts of 5-7

| | RT | ³¹P δ |
|---|---|---|
| [R_{P}] Monothioate 5a | 6.1 | −0.5, 55.4 |
| [S_{P}] Monothioate 5b | 4.8 | −0.3, 56.7 |
| [R_{P},R_{P}] Dithioate 6a | 8.0 | 55.4 |
| [S_{P},R_{P}] Dithioate 6b | 6.2 | 55.9, 57.4 |
| [S_{P},S_{P}] Dithioate 6c | 5.4 | 57.2 |
| [R_{P}] Trithioate 7a | 9.1 | 55.3, 114.1 |
| [S_{P}] Trithioate 7b | 8.0 | 57.2, 114.4 |

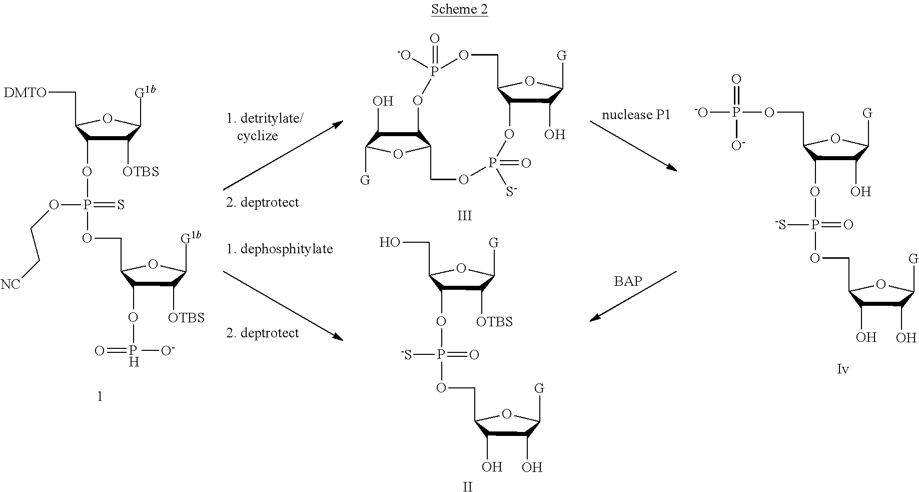

### NMR Characterization

1D ¹H and ³¹P NMR were used at several temperatures to assess the polymorphism of each of the seven thioate analogs, for both the Na⁺ and K⁺ salts. 2D NOESY experiments were also used to assign the syn or anti conformation of the guanine ring relative to the ribose, based on the well-known observation that at low mixing times, syn arrangements give pronounced crosspeaks between the guanine H8 and the ribose H1′, while anti arrangements do not. Further, 2D DOSY (diffusion-ordered spectroscopy) experiments were used to qualitatively evaluate relative size by means of molecular diffusion coefficients. Work with unmodified c-di-GMP in 0.1 M LiCl demonstrated an equilibrium among five different complexes in similar amounts, four of which contain guanine quartets: a bimolecular structure, a pair of tetramolecular complexes made of two parallel guanine quartets (one all syn and one all anti), and a pair of octamolecular intercalated complexes made of four stacked quartets (one all syn and one all anti). In 0.1 M KCl, the two octamolecular complexes dominated the equilibrium. c-di-GMP in 0.1 M NaCl appeared to form similar complexes with stabilities somewhat like those in LiCl, but also showed signs of additional aggregation not seen with the other salts.

Partial ¹H NMR spectra that display the H8 region for both the K⁺ and Na⁺ salts of 5-7 at 30° C. Because the H8 region is the most informative, only that section is described herein. Spectra at lower temperatures (5 and 15° C.) display increased resonances for the less stable higher order complexes (tetra- and octamolecular) relative to the bimolecular structure, but reduced resonances for them at higher temperature (55° C.). ³¹P NMR spectra were also acquired at multiple temperatures. Those resonances were assigned based on their temperature dependence, and relationship with the ¹H NMR spectra using correlations determined from our previous work with unmodified c-di-GMP. (Zhang et al., Journal of the American Chemical Society, 128, 7015-7024 (2006)). Except for 6c and 7b (which displayed unusually poor signal to noise ratio due to extensive aggregation), the ³¹P NMR resonances for the different forms were integrated, and the relative amounts are summarized in Table 2. 2D NOESY plots for the eight samples that display strong crosspeaks characteristic of syn conformations were also plotted.

TABLE 2
Distribution of Complexes at 30° C. Based on Integration of ³¹P NMR Spectra

| | | K⁺ salts | | | Na⁺ salts | | |
|---|---|---|---|---|---|---|---|
| | # [S_{P}] | | % anti | % syn | | % anti | % syn |
| | sulfurs ^{a} | % B ^{b} | T/O ^{c} | T/O ^{c} | % B ^{b} | T/O ^{c} | T/O ^{c} |
| [R_{P}] Monothioate 5a | 0 | 5 | 60 O | 35 O | 50 | 33 T | 17 T |
| [S_{P}] Monothioate 5b | 1 | 2 | 56 O | 42 O | 12 | 88 T | ^{e} |
| [R_{P}, R_{P}] Dithioate 6a | 0 | 7 | 60 O | 33 O | 49 | 35 T | 16 T |
| [S_{P}, R_{P}] Dithioate 6b | 1 | 4 | 76 O | 20 O | 22 | 78 T | ^{e} |
| [S_{P}, S_{P}] Dithioate 6c | 2 | | ^{d} | | | ^{d} | |
| [R_{P}] Trithioate 7a | 1 | 5 | 64 O | 31 O | 26 | 74 T | ^{e} |
| [S_{P}] Trithioate 7b | 2 | | ^{d} | | | ^{d} | |

^{a} number of sulfurs in the [S_{P}] configuration,
^{b} B = Bimolecular structure
^{c} T/O = Tetra/Octamolecular complexes,
^{d} = not determined due to extensive aggregation,
^{e} no syn form was detectable

For all 14 samples at 30° C., the H8 regions of the ¹H NMR spectra and the ³¹P NMR spectra are described below, along with syn or anti determinations from the NOESY plots and, where possible, complex size based on diffusion coefficients in m²/sec×10⁻¹⁰ (D) from the DOSY plots. In work with unmodified c-di-GMP, diffusion coefficients at 30° C. for the bimolecular structure were found to be 3.3-3.7, those for the tetramolecular complexes were in the range of 2.0-2.1, and those for the octamolecular complexes were 1.8-1.9. (Although the mass of the octamolecular complex is twice that of the tetramolecular, its structure is more compact and therefore their diffusion coefficients are more similar than the mass difference would imply.)

Monothioates, K⁺ salts: The ¹H NMR spectrum of the [R_{P}] diastereomer 5a, K⁺ salt displays two separate groups of octamolecular anti H8 resonances (D=1.5-1.9); two separate groups of octamolecular syn H8 resonances (D=1.6-1.9); and no resonances with D near 3.5 for the bimolecular structure. The ³¹P NMR spectrum displays one small resonance near −1 ppm for the phosphate of the bimolecular structure; a larger set of nearby downfield octamolecular syn resonances; and a larger set of nearby upfield octamolecular anti resonances. It has a similar pattern for the thiophosphate resonances around 55 ppm.

The ¹H NMR spectrum of the [Sₚ] diastereomer 5b, K⁺ salt displays two separate complex groups of octamolecular anti H8 resonances (D=1.5-1.7); one large and one small octamolecular syn resonance (D=1.6-1.7); and a small resonance (D=3.4) for the bimolecular structure. The ³¹P NMR spectrum displays one small resonance near −1 ppm for the phosphate of the bimolecular structure; a larger set of nearby downfield octamolecular syn resonances; and a larger set of nearby upfield octamolecular anti resonances. However, the pattern for the thiophosphate resonances is different, with both sets of octamolecular syn and anti resonances appearing upfield of the small resonance near 56 ppm for the bimolecular structure.

Monothioates, Na⁺ salts: The ¹H NMR spectrum of the [R_{P}] diastereomer 5a, Na⁺ salt displays a group of multiple tetramolecular anti H8 resonances (D=2.0-2.2); a group of several tetramolecular syn H8 resonances (D=1.7-2.2); and one large broad H8 resonance for the bimolecular structure (D=3.8). The ³¹P NMR spectrum displays one large resonance near −1 ppm for the phosphate of the bimolecular structure; a smaller set of nearby downfield tetramolecular syn resonances; and a smaller set of nearby upfield tetramolecular anti resonances. It has a similar pattern for the thiophosphate resonances near 55 ppm.

The ¹H NMR spectrum of the [Sₚ] diastereomer 5b, Na⁺ salt displays two separate groups of tetramolecular anti H8 resonances (D=2.1-2.4); no syn H8 resonances (no strong H8-H1′ NOESY crosspeaks); and two separate sharp resonances for the bimolecular structure (D=3.7-3.9). The ³¹P NMR spectrum displays one small resonance near −1 ppm for the phosphate of the bimolecular structure; and a larger set of nearby upfield tetramolecular anti resonances (but no downfield syn resonances). It has a similar pattern for the thiophosphate resonances near 56 ppm.

Summary for monothioates: Because both diastereomers of these monothioates are unsymmetrical molecules, each has two kinds of H8s, and there are different possible orientations of the monomers in any complex. This situation presumably accounts in part for the multiple H8 resonances. In spite of the resulting complexity of the spectra, several conclusions can be noted. 1) For the K⁺ salts, the higher order complexes are present in much larger amounts than for the Na⁺ salts (Table 2). 2) The [S_{P}] diastereomer in the Na⁺ salt shows a larger fraction of higher order complexes than does the [R_{P}] diastereomer (Table 2). 3) The higher order K⁺ thioates primarily have diffusion coefficients consistent with octamolecular complexes (1.5-1.9), while those of the Na⁺ thioates primarily are consistent with tetramolecular complexes (1.8-2.4). 4) While both syn and anti higher order complexes are present in the K⁺ thioates (with more anti than syn), in the Na⁺ thioates, the [S_{P}] diastereomer has only anti complexes, while the [R_{P}] diastereomer has both syn and anti.

Dithioates, K⁺ salts: The ¹H NMR spectrum of the [R_{P},R_{P}] diastereomer 6a, K⁺ salt displays two close octamolecular anti H8 resonances (D=1.8) and a third upfield octamolecular anti H8 resonance (D=1.6); two syn octamolecular H8 resonances (D=1.6-1.7); and no resonances with D near 3.5 for the bimolecular structure. The ³¹P NMR spectrum displays a small monothiophosphate resonance near 55 ppm for the bimolecular structure; a larger set of nearby downfield octamolecular syn resonances; and a larger set of nearby upfield octamolecular anti resonances.

The ¹H NMR spectrum of the [S_{P},R_{P}] diastereomer 6b, K⁺ salt displays several broad complex octamolecular anti H8 resonances (D=1.7-2.5); a large octamolecular syn H8 resonance (D=1.7); and several small resonances for the bimolecular structure (D=3.2-3.4). The ³¹P NMR spectrum displays two small resonances for the monothiophosphates of the bimolecular structure ([R_{P}] near 55 ppm and [S_{P}] near 56 ppm); a larger set of nearby downfield octamolecular syn resonances; and a larger set of nearby upfield octamolecular anti resonances.

The ¹H and ³¹P NMR spectra of the [S_{P},S_{P}] diastereomer 6c, K⁺ salt both exhibit a poor signal to noise ratio with few resonances. This behavior is also seen for the Na⁺ salt of 6c, as well as both salt forms of 7b, all of which have two sulfurs in the [Sₚ] configuration. The spectra are significantly worse at temperatures below 30°, where there are often no resonances, while heating the sample to 55° gives spectral quality comparable to the other samples at this temperature. A possible explanation is that the presence two [Sₚ] sulfurs promotes extensive aggregation such that only a small amount of non-aggregated material is visible at 30° C. The ¹H NMR spectrum displays several residual H8 resonances that are difficult to assign (D=1.4-1.6); and no resonances with D near 3.5 for the bimolecular structure. Even though the NOESY was acquired at 45° C. for this sample, the spectrum was not good enough to assign syn or anti conformations. The ³¹P NMR spectrum, displays a small monothiophosphate resonance near 57 ppm for the bimolecular structure; and a larger set of nearby upfield resonances (but no downfield resonances).

Dithioates, Na⁺ salt: The ¹HNMR spectrum of the [R_{P},R_{P}] diastereomer 6a, Na⁺ salt displays two complex groups of multiple tetramolecular anti 1-18 resonances (D=2.0-2.5); two tetramolecular syn H8 resonances (D=2.1-2.2); and two H8 resonances for the bimolecular structure (D=3.2-3.3) that overlap with the upfield tetramolecular anti resonances but are resolved in the DOSY spectrum. The ³¹P NMR spectrum displays one large monothiophosphate resonance near 55 ppm for the bimolecular structure; a smaller set of nearby downfield tetramolecular syn resonances; and a smaller set of nearby upfield tetramolecular anti resonances.

The ¹H NMR spectrum of the [S_{P},R_{P}] diastereomer 6b, Na⁺ salt displays two broad tetramolecular anti H8 resonances (D=1.9-2.1); no syn H8 resonances; and two sharp H8 resonances for the bimolecular structure (D=3.2-3.4). The ³¹P NMR spectrum displays a set of two monothiophosphate resonances for the bimolecular structure ([R_{P}] near 55 ppm and [S_{P}] near 56 ppm); and one set of nearby upfield tetramolecular anti resonances (but no downfield syn resonances).

The ¹H NMR spectrum of the [S_{P},S_{P}] diastereomer 6c, Na⁺ salt displays primarily one residual H8 resonance that is consistent with the bimolecular structure (D=3.5). The ³¹P NMR spectrum displays one monothiophosphate resonance near 57 ppm for the bimolecular structure; and a set of broad nearby upfield resonances that are difficult to assign.

Summary for dithioates: The [R_{P},R_{P}] and [S_{P},S_{P}] dithioates (6a and 6c) are symmetrical molecules, while the [S_{P},R_{P}] dithioate is not. The presence of multiple H8 resonances for 6a and 6c therefore indicates additional complexity that are unable to define at this time. None-the-less, all four conclusions noted above for the monothioates hold true for these dithioates as well. In particular, diastereomers with one or two sulfurs in the [S_{P}] configuration (5b, 6b, and 6c) tend not to form syn complexes for the Na⁺ salts, but do for the K⁺ salts; and Na⁺ thioates form primarily tetramolecular complexes while K⁺ thioates form primarily octamolecular complexes. In addition, the presence of two sulfurs in the [S_{P}] configuration (6c) results in spectra with a poor signal to noise ratio that may be the result of extensive aggregation.

Trithioates, K⁺ salt: The ¹H NMR spectrum of the [R_{P}] diastereomer 7a, K⁺ salt displays two complex groups of octamolecular anti H8 resonances (D=1.6-1.9); a large and a small octamolecular syn H8 resonance (D=1.6-1.9); and two H8 resonances for the bimolecular structure (D=3.1-3.2). The ³¹P NMR spectrum displays one small monothiophosphate resonance near 56 ppm for the bimolecular structure; a set of larger nearby downfield octamolecular syn resonances; and a set of larger nearby upfield octamolecular anti resonances. It also has one small dithiophosphate resonance near 114 ppm for the bimolecular structure; and two sets of larger nearby upfield octamolecular syn and anti resonances (but no nearby downfield resonances).

The ¹H NMR spectra of the [S_{P}] diastereomer 7b, K⁺ salt displays a group of multiple anti H8 resonances that reflect complexes that are at least as large as octamolecular (D=1.1-1.3); one or two small syn H8 resonances (D=1.6); and no resonances with D near 3.5 for the bimolecular structure. The NOESY was acquired at 45° C. because the signal to noise ratio was somewhat better than that at 30° C. The ³¹P NMR spectrum shows one small monothiophosphate resonance near 58 ppm for the bimolecular structure; and a set of larger nearby upfield syn and anti resonances (but no nearby downfield resonances). It also has one small dithiophosphate resonance near 116 ppm for the bimolecular structure; and a larger set of nearby upfield syn and anti resonances (but no nearby downfield resonances).

Trithioates, Na⁺ salt: The ¹H NMR spectrum of the [R_{P}] diastereomer 7a, Na⁺ salt displays two small tetramolecular anti H8 resonances (D=1.8-1.9); no syn H8 resonances; and two large H8 resonances for the bimolecular structure (D=3.1-3.2). The ³¹P NMR spectrum displays one monothiophosphate resonance near 56 ppm for the bimolecular structure, and a set of nearby upfield tetramolecular anti resonances (but no nearby downfield resonances). It also has a dithiophosphate resonance near 114 ppm for the bimolecular structure, and a set of nearby upfield tetramolecular anti resonances (but no nearby downfield resonances).

The ¹H NMR spectrum of the [S_{P}] diastereomer 7b, Na⁺ salt displays two H8 resonances (D=2.7-2.8) that appear to be anti (no strong NOESY crosspeaks), but their size is difficult to assess. The NOESY was acquired at 45° C. because the signal to noise ratio was somewhat better than that at 30° C. The ³¹P NMR spectrum shows a dithiophosphate resonance near 58 ppm for the bimolecular structure; and a set of nearby upfield anti resonances (but no nearby downfield resonances). It also has one dithiophosphate resonance near 116 ppm for the bimolecular structure; and a set of nearby upfield anti resonances (but no nearby downfield resonances).

Summary for trithioates: The trithioates are both unsymmetrical molecules, each with two kinds of H8s, and so have multiple possible orientations in any complex. The trends noted above also apply to these trithioates. Again, diastereomers with one or two sulfurs in the [S_{P}] configuration (5b, 6b, 6c, 7a, and 7b) tend not to form syn complexes as the Na⁺ salts, but do as the K⁺ salts. Further, all the Na⁺ thioates form primarily tetramolecular complexes while the K⁺ thioates form primarily octamolecular complexes. In addition, the results for the [S_{P}] trithioate support the rationale that the presence of two sulfurs in the [S_{P}] configuration (6c, 7b) results in extensive aggregation.

### General Methods

Pivaloyl chloride was freshly distilled before each use. The amidite coupling reactions were carried out in anhydrous acetonitrile that had been dried over 3 Å molecular sieves. The starting materials for all reactions in dry pyridine were dried by concentration from pyridine three times.

Preparative silica gel chromatography was carried out on pre-packed silica gel flash columns from AnaLogix using gradients of methanol in CH₂Cl₂ containing 0.5% pyridine. Analytical reverse phase HPLC was carried out on a Waters 2960 system, with an Atlantis C18 column, 100 Å, 4.6 mm×50 mm, 3.0 μm. Gradients of acetonitrile and 0.1 M triethylammonium acetate (TEAA) buffer (pH 6.8) were used with a flow rate of 1.0 mL/min. ESI-MS was acquired using a Waters Micromass single quadropole LCZ system. Semi-preparative reverse phase HPLC purification was performed on a Waters Novapak C18 19×300 mm column using gradients of CH₃CN in 0.1 M TEAA (pH 6.8) or a Beckman ultrapore RPSC C3 10×250 mm column using gradients of CH₃CN in 0.1 M diisopropylethyl ammonium acetate (pH 6.8). Desalting of pure samples was performed on a Waters Novapak C18 19×300 mm column using gradients of CH₃CN in 0.1 M ammonium bicarbonate. Sodium and potassium salts of 5-7 were obtained by ion exchange using 10 mL of AG 50W-X2 sulfonic acid resin, which had previously been converted to the Na⁺ or K⁺ forms, respectively.

### NMR

The ¹H and ³¹P NMR spectra were acquired on a Varian Inova 500 MHz spectrometer, with the latter referenced to neat phosphoric acid. The samples were 31 mM cyclic dimer in 0.30 mL H₂O containing 10% D₂O, pH 6.8 (adjusted with HCl or either KOH or NaOH, as appropriate). The thioates were quantified by OD measurement at 260 nm using e=26,100 OD M⁻¹ cm⁻¹.⁸

The NOESY and DOSY spectra were acquired at 30° C. (unless otherwise noted) on a Varian Inova 500 MHz spectrometer. NOESY data were collected by 4096 (t2) times 512 (t1) complex data points with spectral widths of 8000 Hz in both dimensions. The mixing time for NOESY spectra was 150 ms, the number of scans per each t1 increment was 16, and the relaxation delay for each scan was 2 s. The DOSY spectra were collected over a spectral width of 8000 Hz using 16 scans for each of the 256 increments, with a relaxation delay of 2.5 sec and a diffusion delay of 0.1 sec. The gradient pulse strength was arrayed from 400 to 20,000. Both NOESY and DOSY acquisitions used 1 s ¹H presaturation during the relaxation delay to suppress water.

### Syntheses of 2a and 2b

2-N-Isobutyryl-2′-O-tert-butyldimethylsilyl-3′-O—(H-phosphonate)-guanosine, sodium salt (2a). To 2-N-isobutyryl-2′-O-tert-butyldimethylsilyl-5′-O-(4,4′-dimethoxytrityl)-guanosine (3.85 g, 5.0 mmol) dissolved in 15 mL of dry pyridine was added diphenyl phosphite (1.9 mL, 10 mmol, 2 eq). After 15 mM at room temperature the reaction mixture was poured into saturated NaHCO₃ and extracted with CH₂Cl₂ (3×50 mL) The organic layers were concentrated, and the residue was purified by silica gel chromatography. Product fractions were combined, concentrated to dryness, and the residue detritylated by shaking a CH₂Cl₂ solution with NaHSO₄/SiO₂ (1.36 g, 3.0 mmol) for 30 min. After filtration, the filtrate was evaporated with frequent addition of toluene (50 mL×3), the last time with about 100 mL remaining. The bulk of the product was deposited on the wall of the flask, leaving the 4,4′-dimethoxytritanol in solution. Hexane (15 mL) was added to the solution, which was allowed to stand for 30 min. The liquid was decanted and the residue washed with 20 mL 15% hexane in toluene. The residue was then dissolved in CH₂Cl₂, made into a foam, and dried in a desiccator over P₂O₅ to give 2a (2.25 g, 3.7 mmol, 74%). The product was confirmed by LC-MS in negative mode, with m/z (M−1) 530.4 (calcd for C₂₀H₃₃N₅O₈PSI: 530.2).

2-N-Isobutyryl-2′-O-tert-butyldimethylsilyl-3′-O—(H-thiophosphonate)-guanosine, sodium salt (2b). To 2-N-isobutyryl-2′-O-tert-butyldimethylsilyl-5′-O-(4,4′-dimethoxytrityl)-guanosine (3.21 g, 4.1 mmol) dissolved in 15 mL of dry pyridine was added diphenylphosphite (1.6 mL, 8.2 mmol, 2 eq). After 15 min lithium sulfide (Li₂S, 0.94 g, 20.5 mmol, 5 eq) was added. After 40 min, the mixture was poured into saturated NaHCO₃ and extracted with CH₂Cl₂ (3×50 mL). The organic layers were concentrated and the residue was purified by silica gel chromatography. Product fractions were combined, concentrated to dryness and the residue dissolved in CH₂Cl₂. Detritylation occurred on standing overnight and the product was separated from 4,4′-dimethoxytritanol as described for 2a to give the H-thiophosphonate 2b (1.81 g, 2.9 mmol, 71%). The product was confirmed by LC-MS in negative mode, with m/z (M−1) 546.4 (calcd for C₂₀H₃₃N₅O₇PSSi⁻: 546.2).

Synthesis of the [R_{P}] monothiophosphate (c-GpGps) (5a)

2-N-Isobutyryl-2′-O-tert-butyldimethylsilyl-guanosinylyl (3′→5′)-2-N-isobutyryl-2′-O-tert-butyldimethylsilyl-3′-O—(H-phosphonate)-guanosine, sodium salt (3a). To a mixture of phosphoramidite 1 (1.45 g, 1.5 mmol, 1.5 eq) and H-phosphonate 2a (0.61 g, 1.0 mmol) dissolved in 20 mL CH₃CN at room temperature was added pyridinium trifluoroacetate (0.58 g, 3 mmol, 2 eq to 1). The mixture was stirred for 30 min, followed by addition of anhydrous tert-butylhydroperoxide (0.87 mL, 6.25 mmol) in decane. After 30 min, the mixture was poured into aqueous NaHCO₃ and extracted with ethyl acetate. The organic layers were then concentrated and purified to >80% purity by silica gel chromatography. The product fractions were concentrated to dryness, the residue dissolved in CH₂Cl₂ and treated with NaHSO₄/SiO₂(0.91 g), and the product isolated as described above for 2a to give 0.64 g crude 3a as a mixture of two unresolved diastereomers. The 3a mixture was confirmed by LC-MS in negative mode, with m/z (M−1) 1113.0 (calcd for C₄₃H₆₈N₁₁O₁₆P₂Si₂⁻: 1112.4).

Cyclo-2-N-isobutyryl-2′-O-tert-butyldimethylsilyl-P-guanosinylyl-(3′→5′)-2-N-isobutyryl-2′-O-tert-butyldimethylsilyl-[R_{P}]—P-thioguanosinylyl-(3′→5′), sodium salt (4a). To a solution of a portion of crude 3a from above (0.28 g) in 15 mL dry pyridine was added pivaloyl chloride (Pv-Cl, 0.12 mL, 1.0 mmol). After 3 min, elemental sulfur (40 mg, 1.25 mmol) was added, and the reaction was stirred for 40 min. The mixture was then concentrated, evaporated with toluene three times and CH₃CN three times, filtered to remove excess sulfur, and concentrated to dryness to give crude 4a (0.25 g). The residue was not further purified before deprotection, which is described below. 4a in the residue was confirmed by LC-MS in negative mode, with m/z (M−1) 1127.1 (calcd for C₄₃H₆₆N₁₁O₁₅P₂SSi₂⁻: 1126. 4).

Cyclo-P-guanosinylyl (3′→5′)-[R_{P}]—P-thioguanosinylyl-(3′→5′), ammonium salt (c-GpGps [R_{P}], 5a). To the crude 4a from above (0.25 g) was added 4 mL of methylamine in water (40%). After 40 min the mixture was concentrated and evaporated with pyridine three times to remove water. To the residue was added TEA.3HF (2.2 mL, 13.3 mmol) and an additional 1.5 mL TEA. After being heated at 50° C. for 4 hrs, the reaction was quenched with 7 mL isopropyl trimethylsilyl ether. Ethyl ether (10 mL) was added to complete precipitation of 5a. The mixture was allowed to stand overnight, the supernatant was decanted, and the product purified by semi-preparative RP HPLC on a Waters Novapak C18 column using a gradient of 2-20% CH₃CN in 0.1 M TEAA over 90 min. The pure fractions were pooled, lyophilized, and desalted by C18 RP-HPLC using a gradient of CH₃CN in 0.1 M ammonium bicarbonate to give pure 5a (12 μmol, 3% from 2a). 5a was confirmed by LC-MS in negative mode, with m/z (M−1) 705.3 (calcd for C₂₀H₂₃N₁₀O₁₃P₂S⁻: 705.1).

Synthesis of the [R_{P}] (5a) and [S_{P}] (5b) monothiophosphates (c-GpsGp)

2-N-Isobutyryl-2′-O-tert-butyldimethylsilyl-P-thioguanosinylyl (3′→5′)-2-N-isobutyryl-2′-O-tert-butyldimethylsilyl-3′-O—(H-phosphonate)-guanosine, sodium salt (3b). To a mixture of phosphoramidite 1 (1.45 g, 1.5 mmol, 1.7 eq) and H-phosphonate 2a (0.54 g, 0.90 mmol) dissolved in 20 mL CH₃CN at room temperature was added pyridinium trifluoroacetate (0.58 g, 3.0 mmol, 2 eq to 1). After 30 min, elemental sulfur (0.16 g, 5.0 mmol) dissolved in 30 mL 1:1 pyridine:CH₂Cl₂ was added, and the mixture was stirred for 40 min. The mixture was then concentrated, evaporated with toluene three times and CH₃CN three times, filtered to remove excess solid sulfur, and concentrated to dryness. The residue was purified to >80% purity by silica gel chromatography. The product fractions were concentrated to dryness, the residue dissolved in CH₂Cl₂ and treated with NaHSO₄/SiO₂(0.91 g) as described above for 2a to give 0.72 g of crude 3b as a mixture of two partially resolved diastereomers. The 3b mixture was confirmed by LC-MS in negative mode, with m/z (M−1) 1128.9 (calcd for C₄₃H₆₈N₁₁O₁₅P₂SSi₂⁻: 1128.4).

Cyclo-2-N-isobutyryl-2′-O-tert-butyldimethylsilyl-P-thioguanosinylyl (3′→5′)-2-N-isobutyryl-2′-O-tert-butyldimethylsilyl-P-guanosinylyl-(3′→5′), sodium salt (4b) To a solution of a portion of crude 3b from above (0.41 g) in 15 mL dry pyridine was added Pv-Cl (0.17 mL, 1.4 mmol). After 3 min, water (0.10 mL, 5.6 mmol, 4 eq to Pv-Cl) was added, followed by iodine (0.432 g, 1.7 mmol). After 5 min the mixture was poured into aqueous NaHCO₃ containing Na₂SO₃ (0.34 g) and extracted using CH₂Cl₂. The organic layers were concentrated with addition of toluene three times and purified to >80% purity by silica gel chromatography to give crude 4b (0.14 g) as a mixture of two partially resolved diastereomers. The 4b mixture was confirmed by LC-MS in negative mode, with m/z (M−1) 1126.7 (calcd for C₄₃H₆₆N₁₁O₁₅P₂SSi₂⁻: 1126.4).

Cyclo-[R_{P}]—P-thioguanosinylyl (3′→5′)-P-guanosinylyl-(3′→5′), ammonium salt (c-GpsGp [R_{P}], 5a) and cyclo-[S_{P}]—P-thioguanosinylyl (3′→5′)-P-guanosinylyl-(3′→5′), ammonium salt (c-GpsGp [S_{P}], 5b). The crude mixture of two diastereomers of 4b from above (0.14 g) was treated in the same way as the single diastereomer 4a. C18 RP-HPLC using 0.1 M TEAA buffer and CH₃CN gave two major fractions, each containing 5a or 5b as the major component. Fractions from each peak were combined, further purified by C18 RP-HPLC using a different gradient, and then desalted to give pure 5a (18 μmol, 3% from 2a) and 5b (10 μmol, 2% from 2a). 5a had the same LC-MS profile as above, and 5b (with a different retention time, Table 1) was confirmed by LC-MS in negative mode, with m/z (M−1) 705.3 (calcd for C₂₀H₂₃N₁₀O₁₃P₂S⁻: 705.1).

Synthesis of the [R_{P},R_{P}] (6a) and [S_{P},R_{P}] (6b) dithiophosphates (c-GpsGps)

Cyclo-2-N-isobutyryl-2′-O-tert-butyldimethylsilyl-P-thioguanosinylyl (3′→5′)-2-N-isobutyryl-2′-O-tert-butyldimethylsilyl-P-thioguanosinylyl-(3′→5′), sodium salt (4c). To a solution of a portion of crude 3b from above (0.29 g) dissolved in 15 mL dry pyridine was added Pv-Cl (0.10 mL, 0.8 mmol). After 3 min, elemental sulfur (25.6 mg, 0.8 mmol) was added, and the reaction was stirred for 40 min. The mixture was then concentrated, evaporated with toluene three times and CH₃CN three times, filtered to remove excess sulfur, and concentrated to dryness to give crude 4c (0.22 g) as a mixture of two partially resolved diastereomers. The residue was not further purified before deprotection, which is described below. The 4c mixture was confirmed by LC-MS in negative mode, with m/z (M−1) 1142.9 (calcd for C₄₃H₆₆N₁₁O₁₄P₂S₂Si₂⁻: 1142.3).

Cyclo-[R_{P}]—P-thioguanosinylyl (3′→5′)-[R_{P}]—P-thioguanosinylyl-(3′→5′), ammonium salt (c-GpsGps [R_{P},R_{P}], 6a) and cyclo-[S_{P}]—P-thioguanosinylyl (3′→5′)-[S_{P}]—P-thioguanosinylyl-(3′→5′), ammonium salt (c GpsGps [S_{P},R_{P}], 6b). The crude mixture of two diastereomers of 4c from above (0.22 g) was deprotected as described for 4a. C18 RP-HPLC using 0.1 M TEAA buffer and CH₃CN resolved the mixture into two major fractions, each containing 6a or 6b as the major component. The fractions for each diastereomer were pooled, lyophilized and individually further purified by C18 RP-HPLC, and then desalted to give pure 6a (29 μmol, 3% from 2a) and 6b (16 mol, 2% from 2a). These were characterized by LC-MS with different retention times (Table 1) and m/z (M−1) of 721.6 for 6a and 721.5 for 6b (calcd for C₂₀H₂₃N₁₀O₁₂P₂S₂⁻: 721.0).

Synthesis of the [R_{P},R_{P}] (6a), [S_{P},R_{P}] (6b), and [S_{P},S_{P}] (6c) Dithiophosphates (c-GpsGps)

2-N-Isobutyryl-2′-O-tert-butyldimethylsilyl-P-thioguanosinylyl (3′→5′)-2-N-isobutyryl-2′-O-tert-butyldimethylsilyl-3′-O—(H-thiophosphonate)-guanosine, sodium salt 3c. To a mixture of phosphoramidite 1 (5.81 g, 6.0 mmol, 1.5 eq) and H-thiophosphonate 2b (2.52 g, 4.0 mmol) dissolved in 40 mL CH₃CN was added pyridinium trifluoroacetate (2.31 g, 12 mmol, 2 eq to 1). The mixture was stirred for 30 min, followed by addition of elemental sulfur (0.384 g, 12 mmol) dissolved in 30 mL 1:1 pyridine:CH₂Cl₂. After 40 min, the mixture was concentrated and evaporated with toluene three times and CH₃CN three times, filtered to remove excess sulfur, and concentrated to dryness. The residue was purified to >80% purity by silica gel chromatography. The product fractions were concentrated to dryness, the residue dissolved in CH₂Cl₂ and treated with NaHSO₄/SiO₂(2.73 g) as described above for 2a to give 2.33 g of crude 3c as a partially resolved mixture of diastereomers. The 3c mixture was confirmed by LC-MS in negative mode, with m/z (M−1) of 1144.8 (calcd for C₄₃H₆₈N₁₁O₁₄P₂S₂Si₂⁻: 1144.3).

Cyclo-2-N-isobutyryl-2′-O-tert-butyldimethylsilyl-P-thioguanosinylyl (3′→5′)-2-N-isobutyryl-2′-O-tert-butyldimethylsilyl-P-thioguanosinylyl-(3′→5′), sodium salt 4d). To a solution of a portion of crude 3c from above (0.41 g) in 20 mL of dry pyridine was added diphenyl chlorophosphate (DPP-Cl, 0.1 mL, 0.5 mmol) dissolved in 3 mL pyridine. After 20 min, H₂O (12 μL, 0.67 mmol) was added, followed by I₂ (0.42 g, 1.65 mmol). After 5 min the mixture was poured into aqueous NaHCO₃ containing Na₂SO₃ (0.34 g) and extracted with CH₂Cl₂. The organic layers were concentrated with addition of toluene three times and then purified to >80% purity by silica gel chromatography to give crude 4d (0.21 g) as a partially resolved mixture of three diastereomers. The 4d mixture was confirmed by LC-MS in negative mode, with m/z (M−1) of 1142.3 (calcd for C₄₃H₆₆N₁₁O₁₄P₂S₂Si₂⁻: 1142.3).

Cyclo-[Rₚ]-P-thioguanosinylyl (3′→5′)-[R_{P}]—P-thioguanosinylyl-(3′→5′), ammonium salt (c-GpsGps [R_{P},R_{P}], 6a), cyclo- [̅S̅_̅{̅P̅}̅]̅-̅P-thioguanosinylyl (3′→5′)-[R_{P}]—P-thioguanosinylyl-(3′→5′), ammonium salt (c GpsGps [S_{P},R_{P}], 6b), and cyclo-[S_{P}]—P-thioguanosinylyl (3′→5′)-[S_{P}]-P-thioguanosinylyl-(3′→5′), ammonium salt (c-GpsGps [S_{P},S_{P}], 6c). The crude mixture of three diastereomers of 4d from above (0.21 g) was deprotected as described for 4a. C18 RP-HPLC using 0.1 M TEAA buffer and CH₃CN gave three major fractions, each containing 6a, 6b, or 6c as the major component. The fractions containing 6a and 6b were further purified by C18 RP-HPLC, and the fractions containing 6c were further purified on a Beckman C3 column. Desalting gave pure 6a (25 mmol, 4% from 2b), 6b (12 μmol, 2% from 2b), and 6c (4 mmol, 1% from 2b). 6a and 6b had the same LC-MS profiles as above, and 6c (with a different retention time, Table 1) was confirmed by LC-MS in negative mode, with m/z (M−1) 721.4 (calcd for C₂₀H₂₃N₁₀O₁₂P₂S₂⁻: 721.0).

Synthesis of the [R_{P}] (7a) and [S_{P}] (7b) trithiophosphates (c-GpsGpss)

Cyclo-2-N-isobutyryl-2′-O-tert-butyldimethylsilyl-P-thioguanosinylyl (3′→5′)-2-N-isobutyryl-2′-O-tert-butyldimethylsilyl-P-dithioguanosinylyl-(3′→5′), sodium salt (4e). To a solution of a portion of crude 3c from above (0.85 g) dissolved in 20 mL dry pyridine was added DPP-Cl (0.22 mL, 1.05 mmol) dissolved in 3 mL pyridine. After 20 min, elemental sulfur (0.068 g, 2.1 mmol) was added, and the mixture was stirred for 40 min. The mixture was concentrated, evaporated with toluene three times and CH₃CN three times, filtered to remove excess sulfur, and concentrated to dryness. The residue was purified to >80% purity by silica gel chromatography to give crude 4e (0.85 g) as a mixture of two partially resolved diastereomers. The 4e mixture was confirmed by LC-MS in negative mode, with m/z (M−1) 1158.7 (calcd for C₄₃H₆₆N₁₁O₁₃P₂S₃Si₂⁻: 1158.3).

Cyclo-[R_{P}—P-thioguanosinylyl (3′→5′)-P-dithioguanosinylyl-(3′→5′), ammonium salt (c-GpsGpss [R_{P}], 7a) and cyclogS_{P}]—P-thioguanosinylyl (3′→5′)-P-dithioguanosinylyl-(3′→5′), ammonium salt (c-GpsGpss [S_{P}], 7b). A portion of the crude mixture of two diastereomers of 4e from above (0.52 g) was deprotected as described for 4a. C18 RP-HPLC using 0.1 M TEAA buffer and CH₃CN gave two major fractions, each containing 7a or 7b as the major component. The fractions containing 7a were further purified by C18 RP-HPLC, and the fractions containing 7b were further purified on a Beckman C3 column. Desalting gave pure 7a (32 μmol, 4% from 2b) and 7b (6 μmol, 1% from 2b). The products had different retention times (Table 1) and were confirmed by LC-MS in negative mode, with m/z (M−1) 737.4 for 7a and 737.1 for 7b (calcd for C₂₀H₂₃N₁₀O₁₁P₂S₃⁻: 737.0).

### Enzymatic Cleavage Experiments

Preparation of ii. To i (0.2 g, 0.13 mmol) dissolved in 9 mL of dry pyridine and ethylene glycol (0.08 g, 1.3 mmol, 10 eq) was added adamantoyl chloride (0.13 g, 0.65 mmol, 5 eq). After 3 hrs, the reaction was quenched with saturated NaHCO₃ and extracted three times with CH₂Cl₂. The organic layers were combined and concentrated. To the residual oil was added 5 mL of aq 40% methylamine. After 1 hr the mixture was dried by concentration from pyridine three times. To the residue was added TEA.3HF (1.8 mL, 11 mmol, 125 eq to TBS) and 1.4 mL TEA. After 4 hrs at 50° C., the reaction was quenched with 5.4 mL isopropyl trimethylsilyl ether and the product collected by centrifugation. To the residue was added 7 mL of 0.5 M acetic acid. After 18 h the mixture was concentrated, the residue dissolved in H₂O, and the solution washed with ethyl ether three times. The aqueous layers were concentrated, and the residue was purified by semi-preparative RP HPLC.

SVPD cleavage of ii. To 50D (0.20 μmol) of ii dissolved in 250 μL buffer (pH 7.0) containing 100 mM Tris-Cl and 2 mM MgCl₂ was added 0.003 units of SVPDE. The solution was maintained at 37° C., with 10 μL aliquots analyzed by RP-HPLC over 80 h. The percentage of remaining linear dimer was determined by integration of the corresponding peak in the chromatograms.

P1, BAP, and SVPD cleavage of iii. To 6 OD (0.23 μmol) of iii dissolved in 600 uL TEAA buffer (pH 7.0) was added 0.30 units of nuclease P1.

The solution was maintained at 37° C. with 15 μL aliquots analyzed by RP-HPLC to monitor the disapperance of cyclic dimer. After the completion of ring-opening, the solution was heated at 95° C. for 30 min. After cooling to room temperature, 2.0 units of BAP were added, and the removal of 5′-phosphate was monitored by HPLC by injection of 15 μL aliquots. After completion of dephosphorylation, the mixture was heated at 95° C. for 30 min. After cooling to room temperature, 0.004 units of SVPDE were added. The mixture was maintained at 37° C. with 15 μL aliquots analyzed by HPLC over 40 h.

P1 and SVPD cleavage of iii. To 60D (0.23 μmol) of iii dissolved in 600 uL TEAA buffer (pH 7.0) was added 0.30 units of nuclease P1. The solution was maintained at 37° C. with 15 μL aliquots analyzed by RP-HPLC to monitor the disappearance of cyclic dimer. After the completion of ring-opening, the solution was heated at 95° C. for 30 min. After cooling to room temperature, the solution was divided into two equal parts. To one was added 0.001 units of SVPDE. The mixture was maintained at 37° C. with 15 μL aliquots analyzed by HPLC over 47 h.

P1 cleavage of iii. The pH of the second part above was adjusted to between 5 and 6 using acetic acid and 0.25 units of nuclease P1 was added. The solution was maintained at 37° C. with 15 μL aliquots analyzed by HPLC over 50 h.

### Example 2

### One-Flask Syntheses of c-di-GMP and the [R_{P},R_{P}] and [R_{P},S_{P}] Thiophosphate Analogs

An integrated set of reactions and conditions that allow an eight step one-flask synthesis of the protected derivatives of c-di-GMP and the [Rₚ,Rₚ] and [Rₚ,Sₚ] thiophosphate analogs is reported. Deprotection is also carried out as a one-flask procedure, with the final products isolated by crystallization from the reaction mixture. Chromatography is only used for separation of the thiophosphate diastereomers.

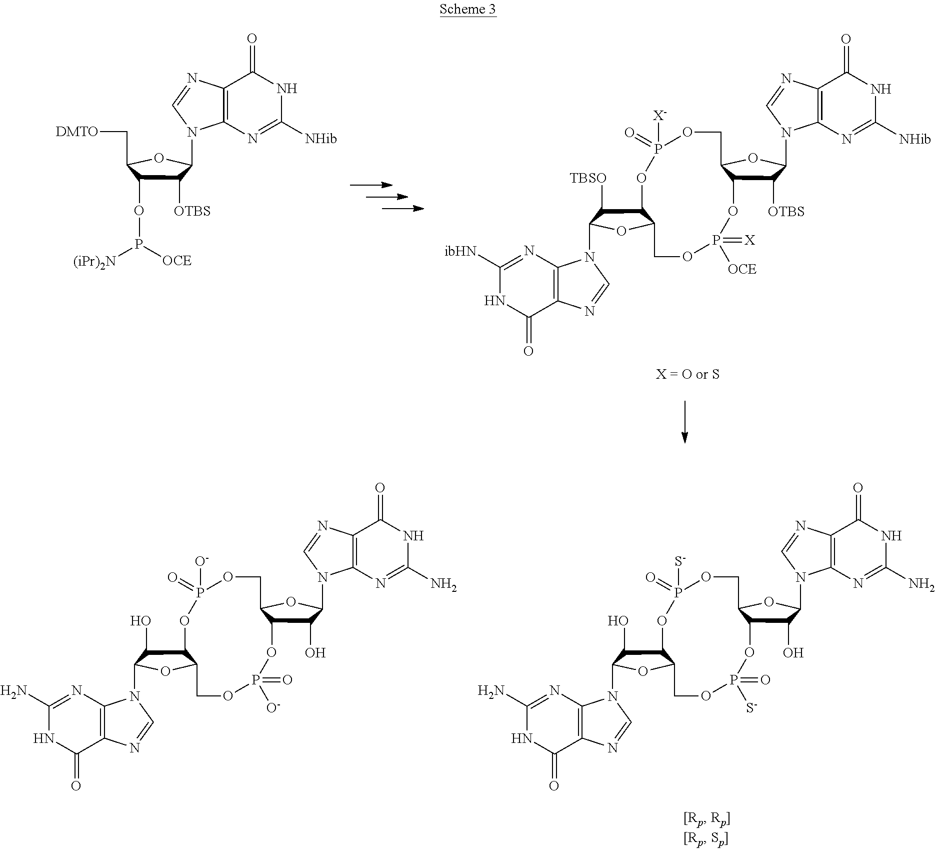

The bacterial signaling molecule, c-di-GMP, 7) is increasingly recognized as having widespread consequences for human health through its multiple roles, which include regulation of biofilm formation as well as virulence. Further, c-di-GMP has now been identified as a specific and high affinity ligand for the GEMM class of bacterial riboswitches that regulate gene expression. Recent crystal structures of the bound riboswitch aptamer suggest a molecular mechanism for the c-di-GMP signaling pathway through formation of a unique helix. These structures demonstrate asymmetrical binding of c-di-GMP, with the guanines deeply buried, but with the ribose phosphate ring partially exposed.

Better synthetic methods for c-di-GMP, as well as analogs and derivatives, are essential to fully explore and define its pathways and molecular mechanisms. A first goal in improving the synthesis of c-di-GMP and its thiophosphate analogs was to develop a fast and convenient route to gram scale quantities from low-cost commercially available starting materials. Previous approaches used a range of chemistries. A common feature of the previous approaches was the small amount of c-di-GMP prepared, with none reporting amounts over 200 mg, and most much less. All rely on reversed-phase HPLC for final purification, and all but the solid-phase route require several silica chromatography steps during the synthesis. The small amount of c-di-GMP obtained limits the opportunity for the preparation of derivatives. Further, as a phosphate triester cyclization does not lend itself to the preparation of thiophosphates. Described in Example 1 is the preparation and characterization of all of the thiophosphate diastereomers of c-di-GMP containing one, two, or three sulfur atoms, but in such modest yields that opportunities for further study are limited, beyond confirming that they showed similar polymorphism to that found for c-di-GMP itself. Thiophosphate analogs of defined stereochemistry are of particular interest as probes of phosphate/metal interaction, and because of the resistance of thiophosphates to enzymatic hydrolysis.

The method for preparation of c-di-GMP and c-di-(guanosine-monothiophosphate) now reported uses the standard commercial N-isobutyryl-2′-O-TBS protected guanosine phosphoramidite, 1, as the common starting material for both of the guanosines incorporated, and was designed such that the reagents used in each step do not interfere with subsequent steps, allowing the syntheses to be carried out as one-flask procedures. Two particular examples of the degree of integration of the reagents are that the dichloroacetic acid used for the first detritylation provides in the next step the pyridinium ion that is the promoter of the amidite coupling reaction, and that the dichloroacetate does not interfere with the H-phosphonate cyclization reaction. Thus, the eight steps to get to the protected cyclic derivatives 5a-c, which are isolated by extraction, are carried out by sequential addition of reagents without isolation of intermediates. A two step, one-flask deprotection procedure is also reported in which the final products are isolated by crystallization, without the need for a fluoride scavenger, ion-exchange, or reversed-phase HPLC. There are no chromatographic steps in preparation of c-di-GMP, 7. The separation of the protected thiophosphate diastereomers 5b and 5c, on a silica column, is the only chromatographic step.

As shown in Scheme 4, the first step is hydrolysis of the first portion of the guanosine phosphoramidite, 1, by treatment with pyridinium trifluoroacetate in acetonitrile containing 2 equiv. water. The hydrolysis takes less than one minute to give the H-phosphonate diester, 2, which is immediately treated with tert-butylamine to remove the cyanoethyl group. This reaction is complete within ten minutes and the solution is then concentrated to remove tert-butylamine prior to detritylation with 3% dichloroacetic acid (DCA) and 10 equiv. of water in methylene chloride. The detritylation requires less than 10 minutes. The water prevents retritylation, which otherwise occurs to a significant extent upon quenching the DCA with pyridine. After changing the solvent to dry acetonitrile, the coupling with 1.3 equiv. of a second portion of 1 to give 3 is complete within two minutes.

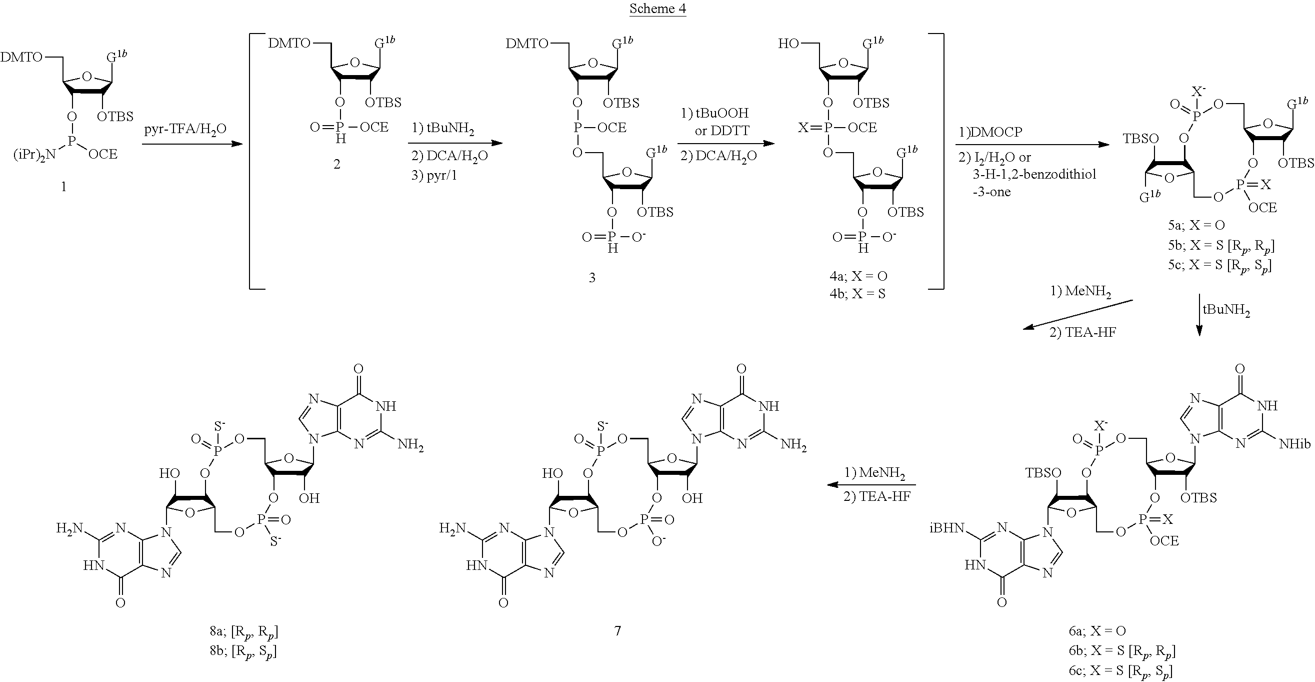

Oxidation using tert-butylhydroperoxide, or sulfurization using 3-((dimethylaminomethylidene)-amino)-3H-1,2,4-dithiazole-5-thione (DDTT), is carried out immediately after the coupling. The oxidation or sulfurization requires no more than 30 minutes. The solution is concentrated to change the solvent back to methylene chloride for the second detritylation, again using DCA/water, to give the linear dimer 4a or 4b. The reaction is quenched by addition of pyridine, followed by concentration to remove methylene chloride and water. Cyclization is then effected by addition of 3-3.5 equiv. of 5,5-dimethyl-2-oxo-2-chloro-1,3,2-dioxaphosphinane (DMOCP) to the pyridine solution of 4a or 4b, and is complete within 5-10 min. Pivaloyl chloride or adamantoyl chloride also work well, but DMOCP gives slightly cleaner results. Significantly, high dilution is not necessary for this H-phosphonate cyclization, which is done at a concentration of 20 mL/mmol. The excess DMOCP is quenched with water, followed by addition of either iodine or 3H-1,2-benzodithiol-3-one. The oxidation or sulfurization requires 5 minutes. Excess iodine is consumed by addition of the mixture to a solution of aqueous sodium bisulfate, to which sodium bicarbonate is added after 10 min. For the thioates the reaction mixture is added directly to a sodium bicarbonate solution.

The protected mixed diester/triester derivatives 5a-c are isolated by extraction of the aqueous mixture with 1:1 diethyl ether and ethyl acetate. The thiophosphate diastereomers 5b and 5c are separated and purified by silica chromatography in yields from 1 of 27 and 23%, respectively. Only two of the three thiophosphate diastereomers are obtained because the H-phosphonate cyclization and sulfurization gives only the Rₚ diastereomer. Treatment of 5a with tert-butylamine in acetonitrile gives 6a, which is readily purified by crystallization from methylene chloride, in 35-45% yield from 1. If desired, 5b and 5c can be similarly converted to 6b and 6c, which can be crystallized from water. The tert-butylamine step, however, is not necessary, as subsequent methylamine treatment efficiently removes the cyanoethyl group.

Full deprotection is carried out in two steps beginning with treatment with a solution of methylamine in ethanol at room temp for 1-2 h. After concentration to dryness, the residue is dissolved in minimal pyridine and treated with TEA.HF at 50° for 60 min. The final products, 7, 8a, or 8b, crystallize from the mixture as the triethylamonium salts upon addition of acetone.

The method reported here is significantly faster and more convenient than other approaches reported to date, allowing facile preparation of gram scale amounts of crystalline c-di-GMP. Of the eight reactions leading to 5a-c, only the min phosphite triester oxidation/sulfurization takes more than ten min. Because of the fast reactions, the overall time for preparation of 5a-c from 1, is 8-10 h, with most of the time spent in concentrations on a rotary evaporator to change solvents. The conversion of 5a to 6a takes only an hour, while the time for crystallization of 6a varies from 1-8 h. The silica separation of 5b and 5c takes most of a day, as does the final deprotection, so that the overall time for 7, or 8a/8b, is about three days with overall yields from 1 of 30% for 7, 19% for 8a, and 17% for 8b.

This route to c-di-GMP is based on the most common commercially available guanosine phosphoramidite, eliminating the need for preparation of this starting material. Moreover, since the phosphoramidites of many other nucleosides, and analogs, are also available, this route is generally applicable to the preparation of a wide variety of other cyclic dinucleotides, including unsymmetrical molecules, as well as their thiophosphate analogs.

### A. General Methods

The amidite coupling reactions and cyclizations were carried out in anhydrous CH₃CN or pyridine, respectively, that had been dried using a silica solvent purification system. Anhydrous reactions were carried out under nitrogen. Preparative silica gel chromatography was carried out on pre-packed silica gel Super Flash columns from Varian using gradients of CH₃OH in CH₂Cl-₂. Analytical reversed phase HPLC was carried out on a Waters 2960 system with a photodiode array detector, with an Atlantis C18 column, 100 Å, 4.6 mm×50 mm, 3.0 μm. Gradients of CH₃CN and 0.1 M triethylammonium acetate buffer (pH 6.8) were used with a flow rate of 1.0 mL/min. Chromatograms in Section D are shown at 250 nm. Low resolution ESI-MS was routinely acquired using a Waters Micromass single quadrupole LCZ system. High resolution ESI-MS for products was obtained from the Washington University Resource for Biomedical and Bio-organic Mass Spectrometry in St. Louis Mo. The ¹H, ¹³C, and ³¹P NMR spectra were acquired on a Varian Inova 500 MHz spectrometer. The ¹H and ¹³C spectra acquired in DMSO-d₆ or D₂O were referenced indirectly to 3-(trimethylsilyl)-1-propane-sulfonic acid, sodium salt, in DMSO-d₆ or D₂O, respectively. The ³¹P NMR spectra acquired in DMSO-d₆ were referenced indirectly to neat H₃PO₄ while those acquired in D₂O were referenced indirectly to 10% phosphoric acid in D₂O.

B. Preparation of 7 on a 5 mmol Scale

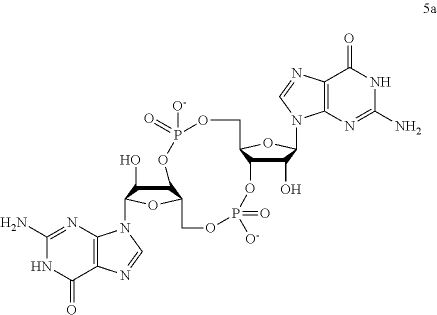

1. One-Flask Synthesis of 5a

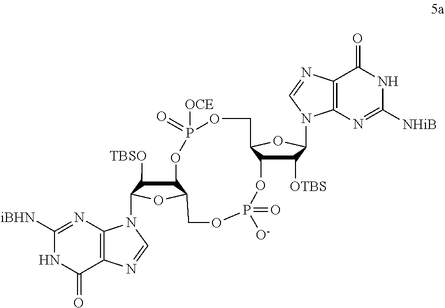

a. Preparation of a dry solution of 1 in CH₃CN. One portion of guanosine phosphoramidite, 1 (6.31 g, 6.5 mmol, 1.3 equiv), was dried three times by concentration from 40 mL portions of CH₃CN, the last time leaving 20 mL. Ten 3 Å molecular sieves were added.

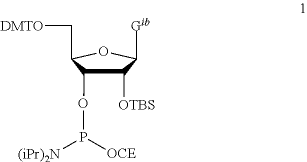

b. Hydrolysis, β-elimination, and detritylation. To a second portion of 1 (4.85 g, 5.0 mmol) dissolved in CH₃CN (25 mL) and H₂O (0.18 mL, 10 mmol, 2 equiv) was added pyridinium trifluoroacetate (1.16 g, 6.0 mmol, 1.2 equiv). After 1 min a 25 mL portion of t-BuNH₂ was added. After 10 min the mixture was concentrated to a foam, the residue was dissolved in a 50 mL portion of CH₃CN, and concentrated again to a foam. This addition of CH₃CN and concentration was repeated one more time. To the residue dissolved in a 60 mL portion of CH₂Cl₂ was added H₂O (0.90 mL, 50 mmol, 10 equiv), followed by a 60 mL portion of 6% dichloroacetic acid in CH₂Cl₂ (44 mmol). After 10 min the reaction was quenched by addition of pyridine (7.0 mL, 87 mmol, 2 equiv rel to DCA). The mixture was then concentrated, and the residue was dissolved in a 40 mL portion of dry CH₃CN and concentrated again. This process was repeated two more times, the last time leaving 12 mL.

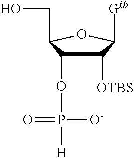

c. Linear coupling, oxidation, and detritylation. To the above solution was added the dried solution of 1 (from step 1a) using a double-tipped needle and nitrogen pressure, followed by two 1 mL rinses of dry CH₃CN. After 2 minutes, anhydrous t-butyl hydroperoxide 5.5 M in decane (2.73 mL, 15 mmol, 3 equiv) was added. After 30 min the solution was cooled in an ice bath, and 1.25 g NaHSO₃ dissolved in 2.5 mL H₂O was added. The ice bath was removed, the mixture was stirred 5 min, and then concentrated to a small volume. The residual oil was dissolved in a 80 mL portion of CH₂Cl₂, followed by H₂O (0.90 mL, 50 mmol, 10 equiv) and then 80 mL 6% dichloroacetic acid in CH₂Cl₂ (58 mmol). After 10 min the reaction was quenched with a 50 mL portion of pyridine. The mixture was concentrated to a small volume, a 150 mL portion of pyridine was added, and the solution was concentrated to 100 mL.

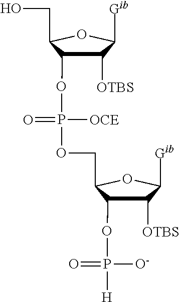

d. Cyclization and oxidation. To the above solution was added 2-chloro-5,5-dimethyl-1,3,2-dioxaphosphorinane-2-oxide (DMOCP, 3.40 g of 95% reagent, 17.5 mmol, 3.5 equiv). After 10 min, the reaction was quenched by addition of H₂O (3.2 mL, 175 mmol, 10 equiv rel to DMOCP), and I₂ (1.65 g, 6.5 mmol, 1.3 equiv) was added immediately. After 5 min the mixture was poured into 700 mL H₂O containing 1.0 g NaHSO₃. After 5 minutes of stirring, 20 g of NaHCO₃ was slowly added.

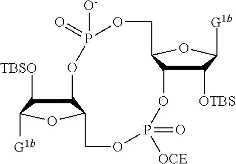

e. Isolation. After 5 min of stirring, the above aqueous solution was partitioned with 800 mL 1:1 EtOAc:Et₂O. Residual gum in the flask was dissolved in 5 mL pyridine and included. The separated aqueous layer was then partitioned with an additional 200 mL of 1:1 EtOAc:Et₂O. The organic layers were combined and concentrated to an oil. Excess pyridine was removed by concentration with three 10 mL portions of toluene.

2. Conversion of 5a to 6a and crystallization of 6a. To the above 5a dissolved in a 25 mL portion of CH₃CN was added a 25 mL portion of t-BuNH₂. After 10 min the mixture was concentrated to a foam, the residue was dissolved in a 25 mL portion of CH₃CN, and concentrated again to a foam. This addition of CH₃CN and concentration was repeated one more time. The residue was then dissolved in a 25 mL portion of CH₃OH for analysis, then filtered, and the filtrate was concentrated to a foam. This foam was dissolved in a 30 mL portion of CH₂Cl₂. Partial crystallization occurred overnight, and full crystallization then took place within minutes upon agitation. 6a was collected by filtration and washed with minimal CH₂Cl₂. The crystals were dried in a dessicator over KOH overnight, giving 2.18 g (1.81 mmol, 36% from 1) of 6a as the t-BuNH₃⁺ salt (C₄₈H₈₆N₁₂O₁₆P₂Si₂). A sample was recrystallized from CH₃OH/CH₂Cl₂, dried, and characterized: mp 191-193° C. dec; the mass of 6a was confirmed by HRMS in negative mode as m/z (M-H) 1057.3393 (calculated for C₄₀H₆₃N₁₀O₁₆P₂Si₂⁻: 1057.3443); UV (CH₃OH) λₘₐₓ 258 nm, sh 281 nm; ¹H NMR (DMSO) 55° C. δ 8.24 (s, 2H), 5.88 (d, J=3 Hz, 2H), 4.93 (br, 2H), 4.38 (br, 2H), 4.14 (br, 2H), 4.10-4.03 (m, 2H), 3.86-3.79 (m, 2H), 2.83 (sep, J=7 Hz, 2H), 1.22 (s, 22H), 1.06 (d, J=7 Hz, 6H), 0.91 (d, J=7 Hz, 6H), 0.84 (s, 18H), 0.14 (s, 6H), 0.07 (s, 6H); ¹³C NMR (DMSO) 55° C. δ (all resonances are singlets) 182.0, 156.4, 149.9, 149.5, 139.5, 122.3, 91.0, 82.4, 77.6, 72.7, 63.3, 52.3, 28.7, 27.4, 20.4, 20.1, 19.4, −2.9, −3.8; ³¹P NMR (DMSO) 55° C. 8-1.87.

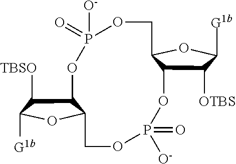

3. Deprotection of 6a and crystallization of 7. To 2.16 g, 1.79 mmol of the above 6a was added 179 mL (1.44 mol, 400 equiv rel to each isobutyryl) CH₃NH₂ in anhydrous ethanol (33% by weight). After 90 min at room temp, the mixture was concentrated to an oil to which a 5 mL portion of pyridine and a 2 mL portion of Et₃N were added. The mixture was concentrated to an oil, and this process was repeated two more times. To the oil was added 4 mL pyridine and the flask with a vent needle was placed in an oil bath at 50° C. Et₃N (25 mL, 180 mmol) and Et₃N.3HF (14.8 mL, 272 mmol F⁻, 75 equiv F⁻ rel to each TBS) were added simultaneously through syringes. The mixture was stirred at 50°, with occasional rotation of the flask at an angle to dissolve all material on the sides. After 1 h, the flask was removed from the oil bath. HPLC grade acetone (200 mL) was immediately added in a slow, steady stream to the stirring mixture. After 10 min of stirring, the crystals were collected by filtration and washed thoroughly 5× with 5 mL portions of acetone. The crystals were dried in a desiccator overnight over KOH, giving 1.32 g (1.48 mmol, 30% from 1) of 7 as the Et₃NH⁺ salt (C₃₂H₅₄N₁₂O₁₄P₂), which was characterized as follows: mp 193-196° C. dec; the mass of 7 was confirmed by HRMS in negative mode as m/z (M-H) 689.0853 (calculated for C₂₀H₂₃N₁₀O₁₄P₂⁻: 689.0876); UV (H₂O) λₘₐₓ 253 nm; ¹H NMR (D₂O) 55° C. δ 8.03 (s, 2H), 5.81 (s, 2H), 5.07 (br, 2H), 4.80 (br, 2H), 4.07-3.96 (m, 2H), 3.13 (q, J=7 Hz, 12H), 1.22 (t, J=7 Hz, 18H); ¹³C NMR (D₂O) 55° C. δ (all resonances are singlets) 159.3 (br), 156.4, 152.1 (br), 139.1, 117.5 (br), 92.8, 83.0, 75.8, 73.0, 65.0, 49.4, 10.8; ³¹P NMR (D₂O) 55° C. δ −0.20.

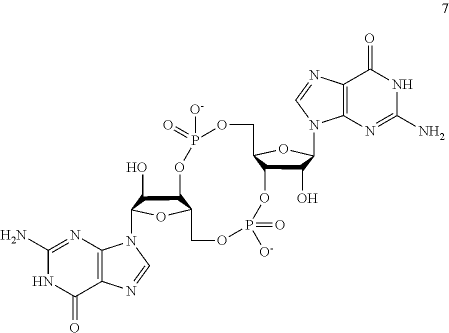

C. Preparation of 8a and 8b on a 3 mmol Scale

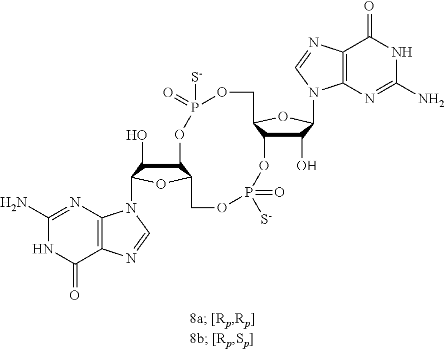

1. One-Flask Synthesis of 5b and 5c

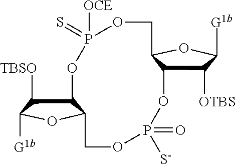

a. Preparation of a dry solution of 1 in CH₃CN. One portion of guanosine phosphoramidite, 1 (3.78 g, 3.9 mmol, 1.3 equiv), was dried three times by concentration from 20 mL portions of CH₃CN, the last time leaving 12 mL. Six 3 Å molecular sieves were added.

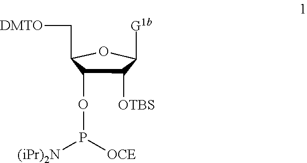

b. Hydrolysis, β-elimination, and detritylation. To a second portion of 1 (2.91 g, 3.0 mmol) dissolved in CH₃CN (15 mL) and H₂O (0.108 mL, 6 mmol, 2 equiv) was added pyridinium trifluoroacetate (0.695 g, 3.6 mmol, 1.2 equiv). After 1 min a 15 mL portion of t-BuNH₂ was added. After 10 min the mixture was concentrated to a foam, the residue was dissolved in a 30 mL portion of CH₃CN, and concentrated again to a foam. This addition of CH₃CN and concentration was repeated one more time. To the residue dissolved in a 36 mL portion of CH₂Cl₂ was added H₂O (0.54 mL, 30 mmol, 10 equiv), followed by a 36 mL portion of 6% dichloroacetic acid in CH₂Cl₂ (26.2 mmol). After 10 min the reaction was quenched by addition of pyridine (4.2 mL, 52 mmol, 2 equiv rel to DCA). The mixture was then concentrated, and the residue was dissolved in a 20 mL portion of dry CH₃CN and concentrated again. This process was repeated two more times, the last time leaving 8 mL.

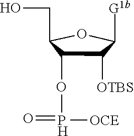

c. Linear coupling, sulfurization, and detritylation. To the above solution was added the dried solution of 1 (from step C1a above) using a dry syringe. After 2 minutes, 3-((N,N-dimethylaminomethylidene)amino)-3H-1,2,4-dithiazole-5-thione (0.677 g, 3.3 mmol, 1.1 equiv) was added. After 30 min the solution was concentrated to a small volume, and the residual oil was dissolved in a 72 mL portion of CH₂Cl₂, followed by H₂O (0.36 mL, 20 mmol, 10 equiv) and 72 mL 6% dichloroacetic acid in CH₂Cl₂ (52.4 mmol). After 10 min the reaction was quenched with a 30 mL portion of pyridine. The mixture was concentrated to a small volume, a 90 mL portion of pyridine was added, and the solution was concentrated to 60 mL.

d. Cyclization, and sulfurization. To the above solution was added 2-chloro-5,5-dimethyl-1,3,2-dioxaphosphorinane-2-oxide (DMOCP, 1.75 g of 95% reagent, 9 mmol, 3 equiv). After 3 min, the reaction was quenched by addition of H₂O (1.6 mL, 90 mmol, 10 equiv rel to DMOCP), and 3H-1,2-benzodithiol-3-one (0.779 g of 97% reagent, 4.5 mmol, 1.5 equiv) was added immediately. After 5 min the mixture was poured into 450 mL H₂O containing NaHCO₃ (12.6 g, 150 mmol).

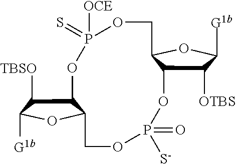

e. Isolation. After 5 min of stirring, the above aqueous solution was partitioned with 450 mL 1:1 EtOAc:Et₂O. The separated aqueous layer was then partitioned with another 150 mL of 1:1 EtOAc:Et₂O. The organic layers were combined and concentrated to an oil. Excess pyridine was removed by concentration with three 10 mL portions of toluene.

2. Separation of 5b and 5c.

The above oil was dissolved in CH₂Cl₂ and applied to a 150 g silica column using a gradient of 0 to 15% CH₃OH in CH₂Cl₂ over 50 min at 10 mL/min. The pure fractions of each diastereomer were combined, concentrated to a foam, and dried in a desiccator over KOH overnight, giving 0.977 g (0.803 mmol, 27% from 1) 5b as the t-BuNH₃⁺ salt (C₄₇H₇₈N₁₂O₁₄P₂S₂Si₂) and 0.853 g (0.700 mmol, 23% from 1) 5c as the t-BuNH₃⁺ salt (C₄₇H₇₈N₁₂O₁₄P₂S₂Si₂). 5b was characterized as follows: the mass of 5b [Rₚ,Rₚ] was confirmed by HRMS in negative mode as m/z (M-H) 1142.3206 (calculated for C₄₃H₆₆N₁₁O₁₄P₂S₂Si₂⁻: 1142.3251); UV (CH₃OH) λₘₐₓ 256 nm, sh 280 nm; ¹H NMR (DMSO) 25° C. δ 12.08 (s, 1H), 12.03 (s, 2H), 11.96 (s, 1H), 8.29 (s, 1H), 8.25 (s, 1H), 6.00 (d, J=7 Hz, 1H), 5.95 (d, J=7 Hz, 1H), 5.41-5.31 (m, 2H), 5.02-4.95 (m, 1H), 4.85-4.79 (m, 1H), 4.60-4.39 (m, 2H), 4.25-4.10 (m, 1H), 3.71-3.64 (m, 1H), 2.99-2.85 m, 2H), 2.72 (sep, J=7 Hz, 1H), 1.20 (s, 3H), 1.14-1.07 (m, 12H), 0.75 (s, 9H), 0.68 (s, 9H), 0.098 (s, 3H), −0.023 (s, 6H), −0.14 (s, 3H); ¹³C NMR (DMSO) 55° C. δ (all resonances are singlets) 181.3, 180.8, 155.5, 150.0, 149.2, 148.6, 139.5, 137.1, 126.6, 125.9, 121.6, 120.6, 118.3, 89.2, 85.8, 82.2, 81.2, 78.7, 74.5, 73.0, 66.7, 64.1, 63.8, 61.9, 47.3, 26.5, 26.1, 19.6, 19.5, 18.2, −2.5, −4.0, −4.4, −4.7, −5.1; ³¹P NMR (DMSO) 25° C. δ 64.86, 57.68. 5c was characterized as follows: the mass of 5c [Rₚ,Sₚ] was confirmed by HRMS in negative mode as m/z (M-H 1142.3202 (calculated for C₄₃H₆₆N₁₁O₁₄P₂S₂Si₂⁻: 1142.3251); UV (CH₃OH) 256 nm, sh 279 nm; ¹H NMR (DMSO) 25° C. δ 12.10 (s, 1H), 12.03 (s, 1H), 12.02 (s, 1H), 11.97 (s, 1H), 8.49 (s, 1H), 8.32 (s, 1H), 6.12 (d, J=8 Hz, 1H), 5.98 (d, J=7 Hz, 1H), 5.37-5.31 (m, 1H), 5.09-5.05 (m, 1H), 4.67-4.52 (m, 3H), 4.33-4.22 (m, 2H), 4.12-4.03 (m, 1H), 3.76-3.68 (m, 1H), 3.00 (t, J=6 Hz, 2H), 2.88 (sep, J=7 Hz, 1H), 2.72 (sep, J=7 Hz, 1H), 1.24 (s, 5H), 1.20 (d, J=7 Hz, 6H), 1.11 (d, J=7 Hz, 6H), 0.70 (s, 8H), 0.65 (s, 9H), 0.067 (s, 3H), 0.059 (s, 3H), −0.028 (s, 3H), −0.19 (s, 3H); ¹³C NMR (DMSO) 55° C. δ (all resonances are singlets) 180.8, 180.8, 155.5, 155.5, 150.1, 149.5, 149.2, 148.8, 139.1, 137.9, 121.5, 120.6, 118.5, 87.5, 85.6, 81.8, 81.2, 78.0, 74.5, 72.7, 72.4, 67.5, 64.6, 64.1, 47.3, 27.9 26.5, 26.1, 19.6, 19.5, 19.4, 19.4, 18.2, −2.5, −4.1, −4.7; ³¹P NMR (DMSO) 25° C. δ 70.92, 56.29.

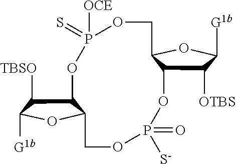

3. Conversion of 5b/c to 6b/c and Crystallization of 6b/c.

Samples of 5b and 5c were individually treated with t-BuNH₂ as described for 5a. The CH₃OH solutions (10 mL) were filtered, and the filtrates were then partially concentrated along with 10 mL H₂O, giving crystalline products as the t-BuNH₃⁺ salts that were dried in a desiccator over KOH for several days. 6b was characterized as follows: mp 205-208° C. dec; the mass of 6b [Rₚ,Rₚ] was confirmed by HRMS in negative mode as m/z (M-H) 1089.2943 (calculated for C₄₀H₆₃N₁₀O₁₄P₂S₂Si₂⁻: 1089.2986); UV (CH₃OH) λₘₐₓ 256 nm, sh 278 nm; ¹H NMR (DMSO) 25° C. δ 8.24 (s, 2H), 5.90 (d, J=4 Hz, 2H), 5.05 (br, 2H), 4.57 (br, 2H), 4.32 (br, 2H), 4.23-4.13 (m, 2H), 3.90-3.81 (m, 2H), 2.82 (sep, J=7 Hz, 2H), 1.22 (s, 6H), 1.08 (d, J=7 Hz, 6H), 0.99 (br, 6H), 0.76 (s, 18H), 0.14 (s, 6H), 0.04 (s, 6H); ¹³C NMR (DMSO) 55° C. (all resonances are singlets unless noted otherwise) 181.9 (br), 156.5 (br), 150.3 (br), 149.7 (br), 139.4, 122.0, 89.7, 82.6, 76.7, 73.5 (d, J_{CP}=6 Hz), 64.4, 52.5, 28.8, 27.3, 20.4, 20.3, 19.3, −2.8, −3.9; ³¹P NMR (DMSO) 25° C. δ 54.65. 6c was characterized as follows: mp 203-206° C. dec; the mass of 6c [Rₚ,Sₚ] was confirmed by HRMS in negative mode as m/z (M-H) 1089.2941 (calculated for C₄₀H₆₃N₁₀O₁₄P₂S₂Si₂⁻: 1089.2986); UV (CH₃OH) λₘₐₓ 255 nm, sh 280 nm; ¹H NMR (DMSO) 25° C. δ 11.97 (br, 2H), 11.87 (br, 2H), 8.31 (s, 1H), 8.28 (s, 1H), 5.98 (d, J=7 Hz, 1H), 5.95 (d, J=7 Hz, 1H), 5.10-5.05 (m, 1H), 5.05-5.00 (m, 1H), 4.75-4.70 (m, 1H), 4.58-4.52 (m, 2H), 4.38-4.34 (m, 1H), 4.34-4.26 (m, 1H), 4.22-4.14 (m, 1H), 3.86-3.79 (m, 1H), 3.74-3.65 (m, 1H), 2.82-2.71 (m, 2H), 1.24 (s, 18H), 1.13-1.08 (m, 13H), 0.68 (s, 18H), 0.12 (s, 3H), 0.09 (s, 3H), −0.01 (s, 3H), −0.02 (s, 3H); ¹³C NMR (DMSO) 55° C. δ (all resonances are singlets unless noted otherwise) 181.8, 181.6, 156.4, 150.8 (d, J_{CP}=26 Hz), 149.7 (d, Jₒ)=13 Hz), 139.3, 138.6, 121.8, 121.6, 88.4, 87.4, 83.66 (d, J_{CP}=7 Hz), 83.1 (d, J_{CP}=7 Hz), 76.3 (d, J_{CP}=5 Hz), 75.8 (d, J_{CP}=5 Hz), 75.5 (d, J_{CP}=5 Hz), 74.1 (d, J_{CP}=8 Hz), 64.1, 52.6, 28.8, 27.2, 27.1, 20.4, 20.4, 20.3, 20.3, 19.3, −2.6, −2.9, −3.9, −4.0; ³¹P NMR (DMSO) 25° C. δ 57.28, 53.81.

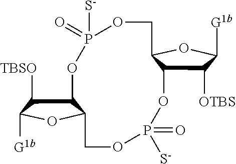

4. Deprotection of 5b and crystallization of 8a. After separation on the column (section C2 above), 5b (0.872 g, 0.717 mmol) was dissolved in 36 mL CH₃NH₂ in anhydrous ethanol (33% by weight) (290 mmol, 200 equiv rel to each isobutyryl). After 90 min at room temp, the mixture was concentrated to an oil, to which a 5 mL portion of pyridine and a 2 mL portion of Et₃N were added. The mixture was concentrated to an oil, and this process was repeated two more times. To the oil was added 2 mL pyridine, and the flask was placed in an oil bath at 50° C. with a vent needle. Et₃N (10 mL, 72 mmol) and Et₃N.3HF (5.9 mL, 108 mmol F⁻, 75 equiv F⁻ rel to each TBS) were added simultaneously through syringes. The mixture was stirred at 50°, with occasional rotation of the flask at an angle to dissolve all material on the sides. After 1 h, the flask was removed from the oil bath. HPLC grade acetone (90 mL) was immediately added in a slow, steady stream to the stirring mixture. After 10 min of stirring, the crystals were collected by filtration and washed thoroughly 5× with 3 mL portions of acetone. The crystals were dried in a desiccator overnight over KOH, giving 0.457 g (0.494 mmol) 8a as the Et₃NH⁺ salt (C₃₂H₅₄N₁₂O₁₂P₂S₂), which was characterized as follows: mp 193-195° C. dec; the mass of 8a [Rₚ,Rₚ] was confirmed by HRMS in negative mode as m/z (M-H) 721.0370 (calculated for C₂₀H₂₃N₁₀O₁₂P₂S₂⁻: 721.0419); UV (H₂O) λₘₐₓ 253 nm; ¹H NMR (D₂O) 55° C. δ 7.97 (s, 2H), 5.87 (s, 2H), 5.05 (br, 2H), 4.74 (br, 2H), 4.05-3.95 (m, 2H), 3.12 (q, J=7 Hz, 12H), 1.21 (t, J=7 Hz, 18H); ¹³C NMR (D₂O) 55° C. δ (all resonances are singlets unless noted otherwise) 160.7, 156.3, 153.1, 139.7, 118.8, 91.7, 82.6 (tₐₚₚ, J_{CP}=10 Hz), 75.9, 73.8 (d, J_{CP}=8 Hz), 66.2 (d, J_{CP}=4 Hz), 49.4, 10.8; ³¹P NMR (D₂O) 55° C. δ 54.47.

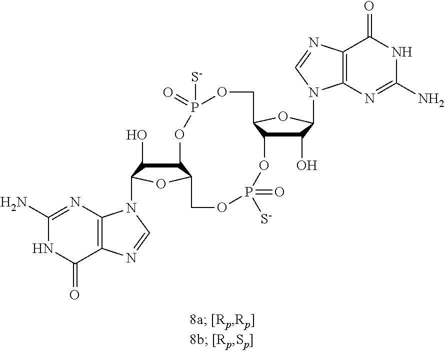

5. Deprotection of 5c and Crystallization of 8b.

After separation on the column (section C2 above), 5c (0.777 g, 0.639 mmol) was dissolved in 32 mL CH₃NH₂ in anhydrous ethanol (33% by weight) (258 mmol, 200 equiv relative to each isobutyryl). After 90 min at room temp, the mixture was concentrated to an oil. To the oil was added a 5 mL portion of pyridine and a 2 mL portion of Et₃N, and the mixture was concentrated to an oil. This process was repeated two more times. To the oil was added 2 mL pyridine and the flask was placed in an oil bath at 50° C. with a vent needle. Et₃N (9 mL, 65 mmol) and Et₃N.3HF (5.2 mL, 96 mmol F⁻, 75 equiv F⁻ rel to each TBS) were added simultaneously through syringes. The mixture was stirred at 50°, with occasional rotation of the flask at an angle to dissolve all material on the sides. After 1 h, the flask was removed from the oil bath. HPLC grade acetone (90 mL) was immediately added in a slow, steady stream to the stirring mixture. After 10 min of stirring, the crystals were collected by filtration and washed thoroughly 5× with 3 mL portions of acetone. The crystals were dried in a desiccator overnight over KOH, giving 0.426 g (0.460 mmol) 8b as the Et₃N⁺ salt (C₃₂H₅₄N₁₂O₁₂P₂S₂), which was characterized as follow: mp 193-196° C. dec; the mass of 8b [Rₚ,Sₚ] was confirmed by HRMS in negative mode as m/z (M-H) 721.0377 (calculated for C₂₀H₂₃N₁₀O₁₂P₂S₂⁻: 721.0419); UV (H₂O) λₘₐₓ 253 nm; ¹H NMR (D₂O) 55° C. δ 8.09 (s, 1H), 7.98 (s, 1H), 5.92 (s, 2H), 5.01 (s, 2H), 4.92 (s, 1H), 4.75 (s, 1H), 4.04-3.96 (m, 2H), 3.12 (q, J=7 Hz, 12H), 1.21 (t, J=7 Hz, 18H); ¹³C NMR (D₂O) 55° C. δ (all resonances are singlets unless noted otherwise) 161.3, 156.4, 153.8 (br), 140.4 (br), 119.2 (br), 91.4, 91.3, 82.8 (tₐₚₚ, J_{CP}=9 Hz), 82.4 (tₐₚₚ, J_{CP}=9 Hz), 75.8, 75.4, 75.0 (d, J_{CP}=4 Hz), 74.2 (d, J_{CP}=8 Hz), 66.1 (d, J_{CP}=3 Hz), 64.8 (d, J_{CP}=6 Hz), 49.4, 10.8; ³¹P NMR (D₂O) 55° C. δ 55.93, 54.65.

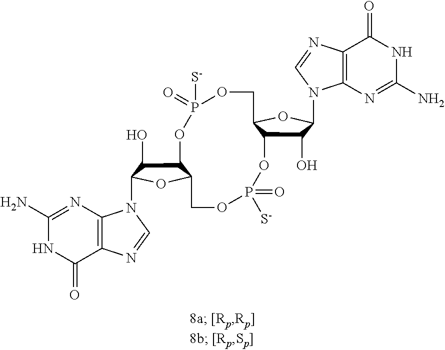

Accordingly, certain embodiments of the present invention provide a method for preparing a compound of formula 2 comprising hydrolyzing a compound of formula 1 to provide the compound of formula 2.

In certain embodiments, the compound of formula 2 is hydrolyzed by treatment with pyridinium trifluoroacetate in acetonitrile in the presence of about 2 equivalents of water.

In certain embodiments, the method further comprises removing the cyanoethyl group from the compound of formula 2 to provide the corresponding compound without the cyanoethyl group.

In certain embodiments, the cyanoethyl group is removed by treatment with tert-butylamine.

In certain embodiments, the method further comprises detritylating the compound without the cyanoethyl group to provide a corresponding compound without a trityl group.

In certain embodiments, the trityl group is removed by treatment with dichloroacetic acid and about 10 equivalents of water in methylene chloride.

In certain embodiments, tert-butylamine is removed prior to treatment with dichloroacetic acid and about 10 equivalents of water in methylene chloride.

In certain embodiments, the method further comprises coupling the compound without a trityl group with a compound of formula 1 to provide a compound of formula 3.

In certain embodiments, the method further comprises quenching the dichloroacetic acid with pyridine, changing the solvent to dry acetonitrile, and coupling with the compound of formula 1 to provide the compound of formula 3.

In certain embodiments, the method further comprises oxidizing the compound of formula 3 to provide the corresponding oxidized formula 3 compound.

In certain embodiments, the compound of formula 3 is oxidized with tert-butylhydrogen peroxide to provide the oxidized formula 3 compound.

In certain embodiments, the method further comprises sulfurizing the compound of formula 3 to provide the corresponding sulfurized formula 3 compound.

In certain embodiments, the compound of formula 3 is sulfurized with 3-((dimethylaminomethylidene)-amino)3H-1,2,4-dithiazole-5-thione (DDTT) to provide the sulfurized formula 3 compound.

In certain embodiments, the method further comprises detritylating the oxidized formula 3 compound to provide a corresponding compound of formula 4a.

In certain embodiments, the trityl group is removed by treatment with dichloroacetic acid and about 10 equivalents of water in methylene chloride.

In certain embodiments, the method further comprises detritylating the sulfurized formula 3 compound to provide a corresponding compound of formula 4b.

In certain embodiments, the trityl group is removed by treatment with dichloroacetic acid and about 10 equivalents of water in methylene chloride.

In certain embodiments, the method further comprises cyclizing the compound of formula 4a to provide a cyclized compound 4a intermediate.

In certain embodiments, the compound of formula 4a is cyclized by treatment with about 3-4 equivalents of DMOCP in pyridine.

In certain embodiments, the method further comprises cyclizing the compound of formula 4b to provide a cyclized compound 4b intermediate.

In certain embodiments, the compound of formula 4a is cyclized by treatment with about 3-4 equivalents of DMOCP in pyridine.

In certain embodiments, the method further comprises quenching with water and treating with iodine or 3H-1,2-benzodithiol-3-one to provide the corresponding compounds of formulae 5a, 5b, and 5c.

In certain embodiments, the method further comprises treating with aqueous sodium bisulfite and subsequently sodium bicarbonate.

In certain embodiments, the method further comprises isolating the compounds of formulae 5a, 5b, and 5c.

Certain embodiments of the present invention provide a method comprising treating a compound of formula 5a with tert-butylamine in acetonitrile to provide compound 6a.

Certain embodiments of the present invention provide a method comprising converting a compound of formula 5b to a compound of formula 6b.

Certain embodiments of the present invention provide a method comprising converting a compound of formula 5c to a compound of formula 6c.

In certain embodiments, the method further comprises crystallizing the resulting compound from water.

In certain embodiments, the method further comprises converting the compound of formula 6a to a compound of formula 7.

In certain embodiments, the method further comprises converting the compound of formula 6b to a compound of formula 8a.

In certain embodiments, the method further comprises converting the compound of formula 6c to a compound of formula 8b.

In certain embodiments, the converting is carried out by treating with a solution of methylamine in methanol, removing the solvent, and adding pyridine, and treating with TEA-HF.

In certain embodiments, the method further comprises treating with a solution of methylamine in methanol, removing the solvent, and adding pyridine, and treating with TEA-HF to provide the corresponding compound of formula 8a or 8b.

All publications, patents and patent applications cited herein are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula 5a, 5b, 6a, 6b, 6c, 7a or 7b 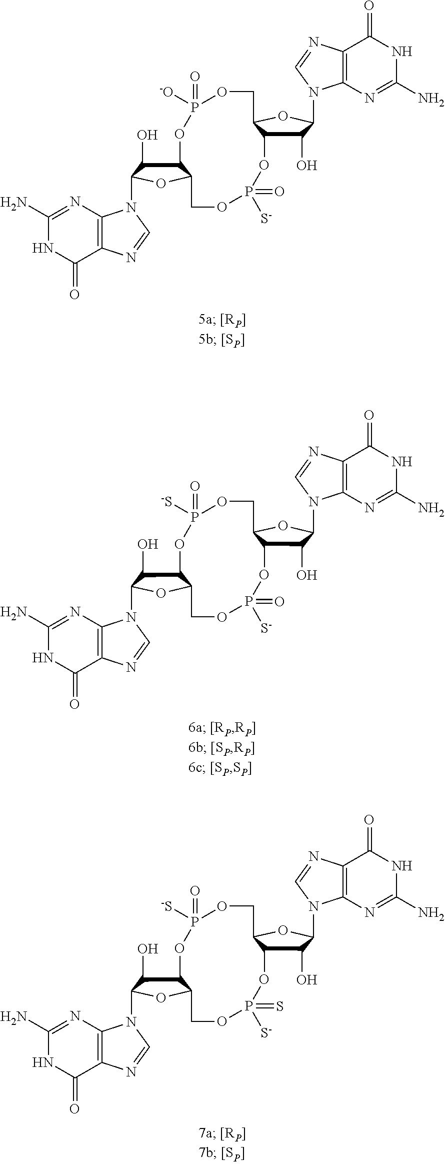
or a salt thereof.

2. The compound of claim 1 that is compound 5a, or a salt thereof.

3. The compound of claim 1 that is compound 5b, or a salt thereof.

4. The compound of claim 1 that is compound 6a, or a salt thereof.

5. The compound of claim 1 that is compound 6b, or a salt thereof.

6. The compound of claim 1 that is compound 6c, or a salt thereof.

7. The compound of claim 1 that is compound 7a, or a salt thereof.

8. The compound of claim 1 that is compound 7b, or a salt thereof.

9. The compound of claim 1, wherein the salt is a potassium salt or a sodium salt.

10. The compound of claim 9, wherein the salt is a sodium salt.

11. The compound of claim 9, wherein the salt is a potassium salt.

12. A composition comprising a compound as described in claim 1, or a salt thereof, and a carrier.

13. The composition of claim 12, wherein the carrier is a pharmaceutically acceptable carrier.

14. A method for preparing a protected derivative of a cyclic dinucleotide or a thiophosphate analog thereof, comprising converting a corresponding first and second phosphoramidite to the protected derivative of the cyclic dinucleotide or a thiophosphate analog thereof, which method comprises, in a single container:
hydrolyzing the first phosphoramidite;
coupling the second phosphoramidite to the hydrolyzed first phosphoramidite to provide a coupled compound;
oxidizing or sulfurizing the coupled compound to provide a linear dimer;
cyclizing the linear dimer;
isolating the protected derivatives;
optionally separating and purifying thiophosphate diastereomers; and
optionally deprotecting the cyclic dinucleotide or a thiophosphate analog thereof to provide the cyclic dinucleotide or a thiophosphate analog thereof.

15. The method of claim 14, comprising separating and purifying thiophosphate diastereomers.

16. The method of claim 14, which comprises deprotecting the cyclic dinucleotide or a thiophosphate analog thereof to provide the cyclic dinucleotide or a thiophosphate analog thereof.

17. The method of claim 14, wherein the coupled compound is oxidized to provide the linear dimer.

18. The method of claim 14, wherein the coupled compound is sulfurized to provide the linear dimer.

19. The method of claim 14, wherein each of the first and second phosphoramidites is individually selected from an adenosine, guanosine, cytidine, uridine, inosine, 5-methyl uridine, 5-bromouridine, 5-iodouridine, 6-thioguanosine, 2-aminopurine, 7-deazadenosine, zebularine, 8-aza-7-deazadenosine, pseudouridine, or 2,6-diaminopurine phosphoramidite, or a 2′-fluoro analog of an adenosine, guanosine, cytidine, or uridine phosphoramidite.

20. The method of claim 19, wherein each of the first and second phosphoramidites are phosphoramidites of the same compound.

21. The method of claim 19, wherein each of the first and second phosphoramidites are phosphoramidites of different compounds.

22. The method of claim 20, wherein the first and second phosphoramidites are guanosine phosphoramidites.

23. The method of claim 14, which method provides a compound of formula 7, or a salt thereof, as a product

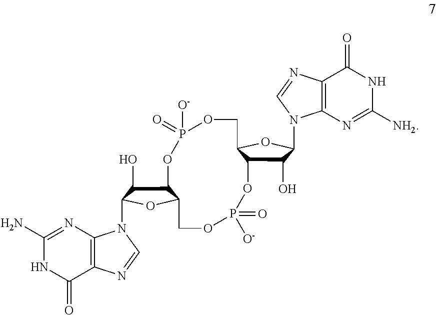

24. The method of claim 14, which method provides a compound of formula 8a or 8b, or a salt thereof, as a product

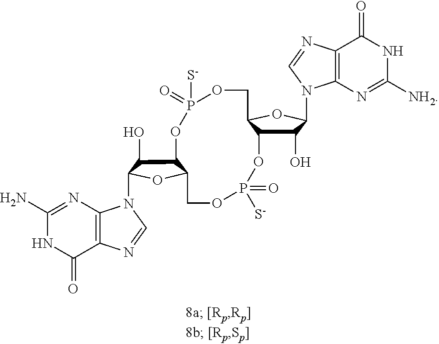

25. The method of claim 14, wherein the container is a flask.

26. A compound prepared according to the method of claim 14.

27-28. (canceled)
